(19) United States
(12) Patent Application Publication
(10) Pub. No.: US 2025/0250292 A1
(43) Pub. Date: Aug. 7, 2025

(54) DELIVERY SYSTEM TO TARGET GRAM-NEGATIVE BACTERIA

(71) Applicant: THE WISTAR INSTITUTE OF ANATOMY AND BIOLOGY, Philadelphia, PA (US)

(72) Inventors: Poli Adi Narayana REDDY, Philadelphia, PA (US); Rishabh SHARMA, Philadelphia, PA (US); Joseph M. SALVINO, Philadelphia, PA (US)

(73) Assignee: THE WISTAR INSTITUTE OF ANATOMY AND BIOLOGY, Philadelphia, PA (US)

(21) Appl. No.: 18/856,686

(22) PCT Filed: Apr. 12, 2023
(86) PCT No.: PCT/US2023/018236

§ 371 Date: Oct. 14, 2024

Related U.S. Application Data
(60) Provisional application No. 63/330,680, filed on Apr. 13, 2022.

(51) Int. Cl. C07F 9/54; A61K 47/54; C07F 9/655

(52) U.S. Cl. CPC C07F 9/5456; A61K 47/54; C07F 9/5442; C07F 9/65522

(57) ABSTRACT

Disclosed herein are compounds of the formulas (I) as well as analogs thereof, wherein the variables are defined herein. Also provided are pharmaceutical compositions thereof. In some aspects, the compounds and compositions provided herein may be used to contact Gram-negative bacteria. Also provided are methods of administering compounds and compositions provided herein to a patient in need thereof, for example, for the treatment or prevention of bacterial infections or diseases.

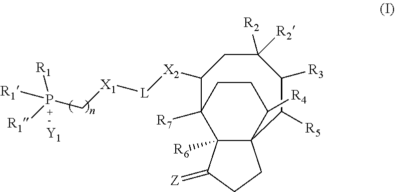

This application claims the benefit of priority to U.S. Provisional Application No. 63/330,680, filed on Apr. 13, 2022, the entire contents of which are hereby incorporated by reference.

### BACKGROUND

### I. Field

This disclosure relates to the fields of biology, pharmacology, medicine, and chemistry. In particular, new compounds, compositions, and methods of treatment related to the treatment of bacterial infection are disclosed.

### II. Description of Related Art

New antibiotics are urgently needed, mainly due to antibiotic resistance among pathogenic bacteria, where some bacterial strains have acquired resistance to nearly all antibiotics. This acquired resistance limits the usage of existing antibiotics, thus creating a significant and ever-growing threat to healthcare (Prestinaci et al., 2015). Drugs targeting gram-negative bacteria represent the majority of the unmet need. The major hurdle against gram-negative pathogens is overcoming the poor cell penetration across the outer membrane (Nikaido, 2003) and efficient efflux of drugs across the inner and outer membrane directly into the external medium (Zgurskaya & Nikaido, 2000; May & Grabowicz, 2018).

Lipophilic cations such as the TPP⁺ moiety have been widely used to target drugs and probes into the mitochondria where it was first described in 1970 (Grinius et al., 1970). The driving force for the accumulation of the lipophilic cation into mitochondria relies on the negative membrane potential maintained across the mitochondrial inner membrane. The gram-negative bacterial cell wall is a rigid and cross-linked matrix of peptidoglycan that is enriched in carboxyl and amino groups. The outer membrane consists of phospholipids on the inside and lipopolysaccharides on the outside in a structure that confers the membrane barrier and efflux activity (Nikaido, 1998). The impermeability of drugs into gram-negative bacteria is rooted in these bacteria's double-membrane cell envelope structure which is highly negatively charged. Furthermore, the gram-negative bacteria possess multidrug efflux systems which enables gram-negative bacteria to survive in a hostile environment. These efflux systems are partially designed to remove positively charged cationic moieties.

Therefore, there remains a need to find new and unique compounds which can penetrate the outer membrane and cell wall of bacteria, especially gram-negative bacteria.

### SUMMARY

In some aspects, the present disclosure provides compounds, pharmaceutical compositions, and methods for their use in the treatment of bacterial infections.

In some aspects, the present disclosure provides compounds comprising a triaryl phosphonium cation and an antibiotic. In some embodiments, the compound further comprises a linker. In some embodiments, the antibiotic is pleuromutilin or a pleuromutilin analog.

In some embodiments, the compounds are further defined as:

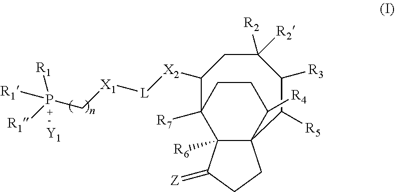

wherein:
   R₁, R₁′, and R₁″ are each independently aryl_{(C≤12)} or substituted aryl_{(C≤12)};
   n is 0, 1, 2, or 3;
   Y₁ is a monovalent anion;
   X₁ and X₂ are each conjugating groups;
   L is a linker group;
   R₂ and R₂′ are hydrogen, alkyl_{(C≤12)}, alkenyl_{(C≤12)}, aryl_{(C≤12)}, aralkyl_{(C≤12)}, or a substitute version thereof;
   R₃ is hydrogen, hydroxy, alkoxy_{(C≤12)}, substituted alkoxy_{(C≤12)}, acyloxy_{(C≤12)}, or substituted acyloxy_{(C≤12)};
   R₄, R₅, R₆, and R₇ are each independently are selected from hydrogen, alkyl_{(C≤12)}, or substituted alkyl_{(C≤12)}; and
   Z is O, S, or NRₐ, wherein Rₐ is hydrogen, alkyl_{(C≤6)}, or substituted alkyl_{(C≤6)};
or a pharmaceutically acceptable salt thereof.

In some embodiments, the compounds are further defined as:

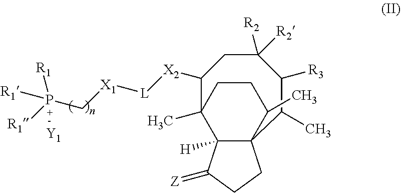

wherein:
R₁, R₁′, and R₁″ are each independently aryl_{(C≤12)} or substituted aryl_{(C≤12)};
n is 0, 1, 2, or 3;
Y₁ is a monovalent anion;
X₁ and X₂ are each conjugating groups;
L is a linker group;
R₂ and R₂′ are hydrogen, alkyl_{(C≤12)}, alkenyl_{(C≤12)}, aryl_{(C≤12)}, aralkyl_{(C≤12)}, or a substitute version thereof;
R₃ is hydrogen, hydroxy, alkoxy_{(C≤12)}, substituted alkoxy_{(C≤12)}, acyloxy_{(C≤12)}, or substituted acyloxy_{(C≤12)}; and
Z is O, S, or NRₐ, wherein Rₐ is hydrogen, alkyl_{(C≤6)}, or substituted alkyl_{(C≤6)};
   or a pharmaceutically acceptable salt thereof.

In some embodiments, the compounds are further defined as:

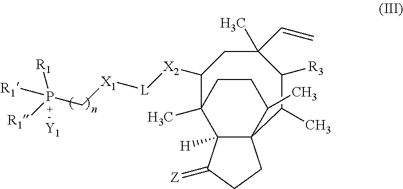

wherein:
R₁, R₁′, and R₁″ are each independently aryl_{(C≤12)} or substituted aryl_{(C≤12)};
n is 0, 1, 2, or 3;
Y₁ is a monovalent anion;
X₁ and X₂ are each conjugating groups;
L is a linker group;
R₃ is hydrogen, hydroxy, alkoxy_{(C≤12)}, substituted alkoxy_{(C≤12)}, acyloxy_{(C≤12)}, or substituted acyloxy_{(C≤12)}; and
Z is O, S, or NRₐ, wherein Rₐ is hydrogen, alkyl_{(C≤6)}, or substituted alkyl_{(C≤6)};
   or a pharmaceutically acceptable salt thereof.

In some embodiments, the compounds are further defined as:

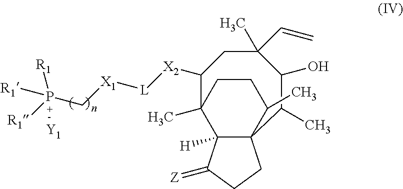

wherein:
R₁, R₁′, and R₁″ are each independently aryl_{(C≤12)} or substituted aryl_{(C≤12)};
n is 0, 1, 2, or 3;
Yᵢ is a monovalent anion;
X₁ and X₂ are each conjugating groups;
L is a linker group; and
Z is O, S, or NRₐ, wherein Rₐ is hydrogen, alkyl_{(C≤6)}, or substituted alkyl_{(C≤6)};
   or a pharmaceutically acceptable salt thereof.

In some embodiments, the compounds are further defined as:

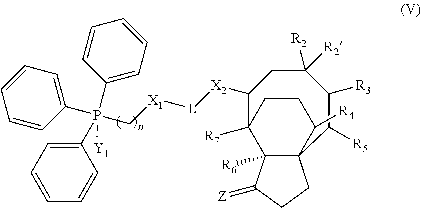

wherein:
n is 0, 1, 2, or 3;
Y₁ is a monovalent anion;
X₁ and X₂ are each conjugating groups;
L is a linker group;
R₂ and R₂′ are hydrogen, alkyl_{(C≤12)}, alkenyl_{(C≤12)}, aryl_{(C≤12)}, aralkyl_{(C≤12)}, or a substitute version thereof;
R₃ is hydrogen, hydroxy, alkoxy_{(C≤12)}, substituted alkoxy_{(C≤12)}, acyloxy_{(C≤12)}, or substituted acyloxy_{(C≤12)};
R₄, R₅, R₆, and R₇ are each independently are selected from hydrogen, alkyl_{(C≤12)}, or substituted alkyl_{(C≤12)}; and
Z is O, S, or NRₐ, wherein Rₐ is hydrogen, alkyl_{(C≤6)}, or substituted alkyl_{(C≤6)};
   or a pharmaceutically acceptable salt thereof.

In some embodiments, the compounds are further defined:

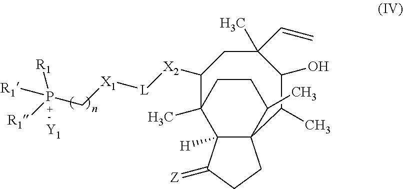

wherein:
R₁, R₁′, and R₁″ are each independently aryl_{(C≤12)} or substituted aryl_{(C≤12)};
n is 0, 1, 2, or 3;
Y₁ is a monovalent anion;
X₁ and X₂ are each conjugating groups;
L is a linker group; and
Z is O, S, or NRₐ, wherein Rₐ is hydrogen, alkyl_{(C≤6)}, or substituted alkyl_{(C≤6)};
   or a pharmaceutically acceptable salt thereof.

In some embodiments, the compounds are further defined as:

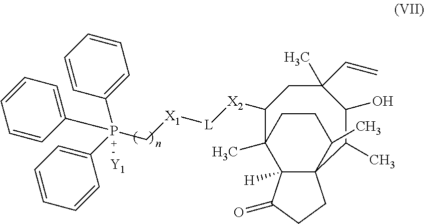

wherein:
   n is 0, 1, 2, or 3;
   Y₁ is a monovalent anion;
   X₁ and X₂ are each conjugating groups; and
   L is a linker group;
      or a pharmaceutically acceptable salt thereof.

In some embodiments, the compounds are further defined as:

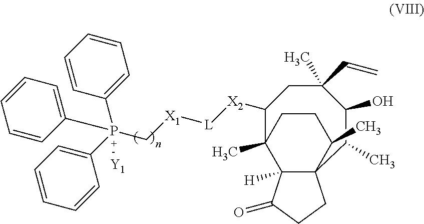

wherein:
   n is 0, 1, 2, or 3;
   Y₁ is a monovalent anion;
   X₁ and X₂ are each conjugating groups; and
   L is a linker group;
or a pharmaceutically acceptable salt thereof.

In some embodiments, R₄ is alkyl_{(C≤12)} or substituted alkyl_{(C≤12)}. In some embodiments, R₄ is alkyl_{(C≤12)} such as methyl. In some embodiments, R₅ is alkyl_{(C≤12)} or substituted alkyl_{(c≤12)}. In some embodiments, R₅ is alkyl_{(C≤12)} such as methyl. In some embodiments, R₆ is hydrogen. In some embodiments, R₇ is alkyl_{(C≤12)} or substituted alkyl_{(C≤12)}. In some embodiments, R₇ is alkyl_{(C≤12)} such as methyl.

In some embodiments, R₂ is alkyl_{(C≤12)} or substituted alkyl_{(C≤12)}. In some embodiments, R₂ is alkyl_{(C≤12)} such as methyl. In some embodiments, R₂′ is alkenyl_{(C≤12)} or substituted alkenyl_{(C≤12)}. In some embodiments, R₂′ is alkenyl_{(C≤12)} such as ethenyl. In some embodiments, R₃ is hydroxy. In some embodiments, Z is O.

In some embodiments, R₁ is aryl_{(C≤12)} or substituted aryl_{(C≤12)}. In some embodiments, R₁ is aryl_{(C≤12)} such as phenyl. In some embodiments, R₁′ is aryl_{(C≤12)} or substituted aryl_{(C≤12)}. In some embodiments, R₁′ is aryl_{(C≤12)} such as phenyl. In some embodiments, R₁″ is aryl_{(C≤12)} or substituted aryl_{(C≤12)}. In some embodiments, R₁″ is aryl_{(C≤12)} such as phenyl.

In some embodiments, n is 0, 1, or 2. In some embodiments, n is 0. In other embodiments, n is 1. In other embodiments, n is 2. In some embodiments, Y₁ is a halide such as bromide or chloride.

In some embodiments, X₁ is a conjugating group selected from: alkanediyl_{(C≤12)}, substituted alkanediyl_{(C≤12)}, alkenediyl_{(C≤12)}, substituted alkenediyl_{(C≤12)}, arenediyl_{(C≤12)}, substituted arenediyl_{(C≤12)}, —C(O)—, —C(O)O—, —OC(O)—, —C(O)NR_{b}—, —NR_{b}C(O)—, —OC(O)O—, —NR_{b}C(O)NR_{b}—, —S(O)ₓ—, —OS(O)ₓO—, —OS(O)ₓ—, —S(O)ₓO—, and a combination thereof; wherein: x is 0, 1, or 2; and R_{b} is hydrogen, alkyl_{(C≤6)}, or substituted alkyl_{(C≤6)}. In some embodiments, X₁ is a conjugating group selected from: alkanediyl_{(C≤12)}, substituted alkanediyl_{(C≤12)}, arenediyl_{(C≤12)}, substituted arenediyl_{(C≤12)}, —C(O)—, —C(O)O—, —OC(O)—, —C(O)NR_{b}—, —NR_{b}C(O)—, and a combination thereof. In some embodiments, X₁ is a conjugating group is a group comprising an arenediyl_{(C≤12)} or substituted arenediyl_{(C≤12)} and —C(O)O— or —OC(O)—. In some embodiments, X₁ is an arenediyl_{(C≤12)} and —C(O)O—. In some embodiments, X₁ is benzenediyl and —C(O)O— such as —C₆H₄—C(O)O—.

In some embodiments, X₂ is a conjugating group selected from: alkanediyl_{(C≤12)}, substituted alkanediyl_{(C≤12)}, alkenediyl_{(C≤12)}, substituted alkenediyl_{(C≤12)}, arenediyl_{(C≤12)}, substituted arenediyl_{(C≤12)}, —C(O)—, —C(O)O—, —OC(O)—, —C(O)NR_{b}—, —NR_{b}C(O)—, —OC(O)O—, —NR_{b}C(O)NR_{b}—, —S(O)ₓ—, —OS(O)ₓO—, —OS(O)ₓ—, —S(O)ₓO—, and a combination thereof; wherein: x is 0, 1, or 2; and R_{b} is hydrogen, alkyl_{(C≤6)}, or substituted alkyl_{(C≤6)}. In some embodiments, X₂ is a conjugating group selected from: —C(O)—, —C(O)O—, —OC(O)—, —C(O)NR_{b}—, —NR_{b}C(O)—, —OC(O)O—, and —NR_{b}C(O)NR_{b}—. In some embodiments, X₂ is a conjugating group selected from: —C(O)—, —C(O)O—, —OC(O)—, and —OC(O)O—. In some embodiments, X₂ is —C(O)O— or —OC(O)—.

In some embodiments, L is a linking group selected from: a covalent bond, alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, alkynediyl_{(C≤12)}, substituted alkynediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, a polypeptide comprising from about 1-100 amino acids, a polyethylene oxide, a polypropylene oxide, a methacrylate polymer, a polyester polymer, a polyamide, a polyamine, —NR_{c}—, —O—, —S—, —C(O)—, —C(O)O—, —OC(O)—, —C(O)NR_{c}—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof; wherein: y is 0, 1, or 2; and R_{c} is hydrogen, alkyl_{(C≤6)}, or substituted alkyl_{(C≤6)}. In some embodiments, L is a linking group selected from: a covalent bond, alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, alkynediyl_{(C≤12)}, substituted alkynediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, or a combination thereof. In some embodiments, L is a linking group selected from: a covalent bond, alkanediyl_{(C≤12)}, substituted alkanediyl_{(C≤12)}, cycloalkanediyl_{(C≤12)}, substituted cycloalkanediyl_{(C≤12)}, arenediyl_{(C≤12)}, substituted arenediyl_{(C≤12)}, or a combination thereof. In some embodiments, L is a linker group selected from: a covalent bond, alkanediyl_{(C≤12)}, or substituted alkanediyl_{(C≤12)}. In some embodiments, L is alkanediyl_{(C≤12)} or substituted alkanediyl_{(C≤12)}. In some embodiments, L is alkanediyl_{(C≤12)} such as —CH₂—.

In other embodiments, L is a linker group selected from: a covalent bond, alkanediyl_{(C≤12)}, substituted alkanediyl_{(C≤12)}, cycloalkanediyl_{(C≤12)}, substituted cycloalkanediyl_{(C≤12)}, heterocycloalkanediyl_{(C≤12)}, substituted heterocycloalkanediyl_{(C≤12)}, —NRₑ—, —O—, —S—, —C(O)—, —C(O)O—, —OC(O)—, —C(O)NR_{c}—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof. In some embodiments, L is a linker group selected from: alkanediyl_{(C≤12)}, substituted alkanediyl_{(C≤12)}, cycloalkanediyl_{(C≤12)}, substituted cycloalkanediyl_{(C≤12)}, heterocycloalkanediyl_{(C≤12)}, substituted heterocycloalkanediyl_{(C≤12)}, —NR_{c}—, —O—, —S—, or a combination thereof. In some embodiments, L comprises an alkanediyl_{(C≤12)}, substituted alkanediyl_{(C≤12)}, a cycloalkanediyl_{(C≤12)}, substituted cycloalkanediyl_{(C≤12)}, and —S—. In some embodiments, L is —X₃—X₄—X₅—; wherein: X₃ is cycloalkandiyl_{(C≤12)} or substituted cycloalkanediyl_{(C≤12)}; X₄ is —S—, and X₅ is alkanediyl_{(C≤12)} or substituted alkanediyl_{(C≤12)}. In some embodiments, X₃ is substituted cycloalkanediyl_{(C≤12)} such as 4-aminocyclohexanediyl. In some embodiments, X₅ is alkanediyl_{(C≤12)} such as methylene.

In some embodiments, the compounds are further defined as:

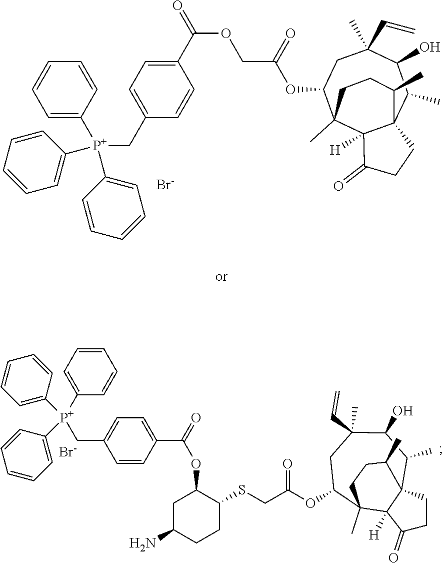 [SMILES: C=C[C@]1(C)C[C@@H](OC(=O)COC(=O)c2ccc(C[P+](c3ccccc3)(c3ccccc3)c3ccccc3)cc2)[C@]2(C)CC[C@@H](C)[C@@]3(CCC(=O)[C@@H]23)[C@@H](C)[C@@H]1O ; [Br-] ; C=C[C@]1(C)C[C@@H](OC(=O)CS[C@@H]2CC[C@@H](N)C[C@H]2OC(=O)c2ccc(C[P+](c3ccccc3)(c3ccccc3)c3ccccc3)cc2)[C@]2(C)CC[C@@H](C)[C@@]3(CCC(=O)[C@@H]23)[C@@H](C)[C@@H]1O ; [Br-]]

or a pharmaceutically acceptable salt thereof.

In another aspect, the present disclosure provides compounds of the formula:

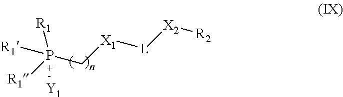

wherein:
R₁, R₁′, and R₁″ are each independently aryl_{(C≤12)} or substituted aryl_{(C≤12)};
n is 0, 1, 2, or 3;
Yᵢ is a monovalent anion;
X₁ and X₂ are each independently conjugating groups;
L is a linker group; and
R₂ is a fluorophore;
   or a pharmaceutically acceptable salt thereof.

In some embodiments, R₁, R₁′, and R₁″ are each the same aryl_{(C≤12)} or substituted aryl_{(C≤12)}. In some embodiments, R₁, R₁′, and R₁″ are each phenyl. In some embodiments, n is 0 or 1. In some embodiments, L is a covalent bond.

In some embodiments, X₁ is alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, alkynediyl_{(C≤12)}, substituted alkynediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, a polypeptide comprising from about 1-100 amino acids, a polyethylene oxide, a polypropylene oxide, a methacrylate polymer, a polyester polymer, a polyamide, a polyamine, —NR_{c}—, —O—, —S—, —C(O)—, —C(O)O—, —OC(O)—, —C(O)NR_{c}—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof; wherein: y is 0, 1, or 2; and R_{c} is hydrogen, alkyl_{(C≤6)}, or substituted alkyl_{(C≤6)}. In some embodiments, X₁ is, —NR_{c}—, —O—, —S—, —C(O)—, —C(O)O—, —OC(O)—, —C(O)NR_{c}—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof; wherein: y is 0, 1, or 2; and R_{c} is hydrogen, alkyl_{(C≤6)}, or substituted alkyl_{(C≤6)}. In some embodiments, X₁ is —C(O)—.

In some embodiments, X₂ is alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, alkynediyl_{(C≤12)}, substituted alkynediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, a polypeptide comprising from about 1-100 amino acids, a polyethylene oxide, a polypropylene oxide, a methacrylate polymer, a polyester polymer, a polyamide, a polyamine, —NR_{c}—, —O—, —S—, —C(O)—, —C(O)O—, —OC(O)—, —C(O)NR_{c}—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof; wherein: y is 0, 1, or 2; and R_{c} is hydrogen, alkyl_{(C≤6)}, or substituted alkyl_{(C≤6)}. In some embodiments, X₂ is, —NR_{c}—, —O—, —S—, —C(O)—, —C(O)O—, —OC(O)—, —C(O)NR_{c}—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof; wherein: y is 0, 1, or 2; and R_{c} is hydrogen, alkyl_{(C≤6)}, or substituted alkyl_{(C≤6)}. In some embodiments, X₂ is —O—.

In some embodiments, the compounds are further defined as:

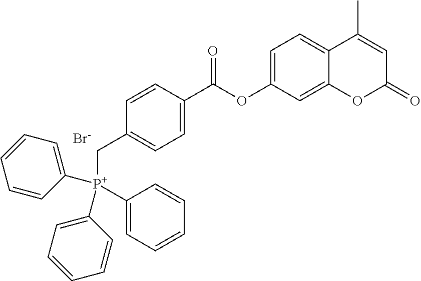 [SMILES: Cc1cc(=O)oc2cc(OC(=O)c3ccc(C[P+](c4ccccc4)(c4ccccc4)c4ccccc4)cc3)ccc12 ; [Br-]]

or a pharmaceutically acceptable salt thereof.

In still yet another aspect, the present disclosure provides pharmaceutical compositions comprising:
(A) a compound described herein; and
(B) an excipient.

In some embodiments, the pharmaceutical compositions are formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion. In some embodiments, the pharmaceutical compositions are formulated as a unit dose.

In still yet another aspect, the present disclosure provides methods of treating a disease or disorder in a patient in need thereof comprising administering a therapeutically effective amount of a compound or composition described herein. In some embodiments, the disease or disorder is an infection. In some embodiments, the infection is a bacterial infection. In some embodiments, the bacterial infection is an infection of a gram positive bacteria such as a Staphylococcus bacteria. In some embodiments, the Staphylococcus bacteria is a Staphylococcus aureus bacteria.

In other embodiments, the bacterial infection is an infection of a gram negative bacteria. In some embodiments, the gram negative bacteria is an Escherichia bacteria such as Escherichia coli bacteria. In other embodiments, the gram negative bacteria is an Klebsiella bacteria such as Klebsiella pneumoniae bacteria. In some embodiments, the methods further comprise administering a second therapeutic agent. In some embodiments, the second therapeutic agent is an antibiotic.

In some embodiments, the methods comprise administering the compound or composition once. In other embodiments, the methods comprise administering the compound or composition two or more times.

In another aspect, the present disclosure provides methods of inhibiting the growth of a bacterium comprising contacting the bacterium with a compound or composition described herein. In some embodiments, the methods are performed in vitro. In other embodiments, the methods are performed in vivo.

In some embodiments, the bacterial infection is an infection of a gram positive bacteria such as a Staphylococcus bacteria. In some embodiments, the Staphylococcus bacteria is a Staphylococcus aureus bacteria.

In other embodiments, the bacterial infection is an infection of a gram negative bacteria. In some embodiments, the gram negative bacteria is an Escherichia bacteria such as Escherichia coli bacteria. In other embodiments, the gram negative bacteria is an Klebsiella bacteria such as Klebsiella pneumoniae bacteria.

In still another aspect, the present disclosure provides methods of killing a bacterium comprising contacting the bacterium with a compound or composition described herein. In some embodiments, the methods are performed in vitro. In other embodiments, the methods are performed in vivo.

In some embodiments, the bacterial infection is an infection of a gram positive bacteria such as a Staphylococcus bacteria. In some embodiments, the Staphylococcus bacteria is a Staphylococcus aureus bacteria.

In other embodiments, the bacterial infection is an infection of a gram negative bacteria. In some embodiments, the gram negative bacteria is an Escherichia bacteria such as Escherichia coli bacteria. In other embodiments, the gram negative bacteria is an Klebsiella bacteria such as Klebsiella pneumoniae bacteria.

It is contemplated that any method or composition described herein can be implemented with respect to any other method or composition described herein. For example, a compound synthesized by one method may be used in the preparation of a final compound according to a different method.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description. Note that simply because a particular compound is ascribed to one particular generic formula doesn't mean that it cannot also belong to another generic formula.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1A-B—Comparison of anti-bacterial toxicity between pleuromutilin and pleuromutilin-TPP+ (AP-8-159B) in (A) gram-positive (S. aureus 49120) and (B) gram-negative (E. coli BL21 (DE3)). AP-8-108 is the unconjugated TPP+ linker.

FIG. 2—Comparison of anti-bacterial toxicity between pleuromutilin and pleuromutilin-TPP+ (AP-8-159B) in gram-positive (S. aureus 46413). AP-8-108 is the unconjugated TPP+ linker.

FIG. 3—Inhibition curve of TPP⁺ linkers in MRC5 cell lines.

FIG. 4—Inhibition curve of AP-8-159B in MRC5 cell lines.

FIG. 5—OD₆₀₀ of samples of AP-8-159B compared to pleuromutilin and the TPP⁺ linker AP-8-108 in an E. coli clinical strain E. coli MG1655.

FIG. 6—OD₆₀₀ of samples of AP-8-159B compared to pleuromutilin and the TPP⁺ linker AP-8-108 in a Klebsiella strain.

### DESCRIPTION OF ILLUSTRATIVE EMBODIMENTS

Provided herein are compounds and compositions that may be used to enter bacterial cells and are thus useful in the treatment in a variety of diseases related to bacterial infection. In some embodiments, these compounds may be used to treat bacterial infections caused by gram-negative bacteria. These compounds may be used as disclosed herein or may be modified to incorporate additional or alternate antibiotic moieties.

Thus, compounds disclosed herein were designed to use the positively charged lipophilic cation (i.e. TPP⁺) (Murphy, 2008) to permeabilize the membrane, interact with the negatively charged phospholipids, and mediate cell-entry. The conjugates were designed with hydrolysable linkers so that the TPP⁺ moiety would dissociate from the antibiotic which would be retained inside the pathogen. This led to the design of a molecule to demonstrate feasibility by incorporation of the lipophilic cation TPP⁺ moiety into an existing ribosome inhibitor to enable the latter to accumulate inside a gram-negative cell. These and other aspects of the disclosure are described in detail below.

Pleuromutilin and similar macrocycles was first discovered in the basidiomycete Pleurotus mutilus (Kavanagh et al., 1951) and is an inhibitor of ribosomal protein synthesis. The pleuromutilins inhibit protein synthesis by binding to the central part of domain V of the 50S ribosomal subunit at the peptidyl transferase center. This prevents the correct positioning of the CCA ends of the tRNAs for the peptide transfer in the A- and P-site thus inhibiting peptide bond formation (Högenauer, 1975; Högenauer & Ruf, 1981). Derivatives such as tiamulin and valnemulin are two well established derivatives in veterinary medicine for oral and intramuscular administration. Lefamulin, a semi-synthetic pleuromutilin compound highly active against multi-resistant pathogens, is a promising antibiotic recently approved by the US FDA for the treatment of community-acquired bacterial pneumonia (McCarthy, 2021). Pleuromutilins typically have potent activity against gram-positive, some gram-negative pathogens, and are unaffected by resistance compared to other major antibiotic classes such as macrolides, fluoroquinolones, tetracyclines, beta-lactam antibiotics, and others which is attributed to its unique and highly specific mode of action (Paukner & Riedl, 2017; Eyal et al., 2016). However, known ribosomal resistance mechanisms have been reported (Long et al., 2006; Gentry et al., 2007). Lefamulin notably lacks activity against most anaerobes as well as Pseudomonas aeruginosa, Acinetobacter baumannii, and E. faecalis (McCarthy, 2021). However, it was suggested that pleuromutilins should possess antibacterial activity against these strains since coupled in vitro transcription-translation assay results showed inhibition of the bacterial translation in these organisms (Paukner & Riedl, 2017). This suggests that the drug has poor exposure in these gram-negative pathogens either due to poor cell permeability or because the drug is rapidly effluxed out of the cell before it can elicit its antibiotic effect. Research at Nabriva revealed that the intrinsic resistance of Enterobacteriaceae is caused by the efflux of pleuromutilins mediated by the AcrAB-TolC efflux pump (Paukner & Riedl, 2017). This is supported by the fact that AcrABToIC deficient E. coli strains were susceptible to pleuromutilins (Paukner & Riedl, 2017) and that the minimum inhibitory concentration (MIC) values against Enterobacteriaceae were significantly reduced by the addition of efflux pump inhibitors (e.g., PAPN). Therefore, pleuromutilin seemed like an excellent antibiotic to use to study the feasibility of a pleuromutilin-TPP⁺ conjugate to compare side-by-side with pleuromutilin as a means to overcome its limitations.

### I. Compounds and Formulations Thereof

### A. Compounds

The compounds of the present disclosure are shown, for example, above, in the summary of the invention section, the Examples section, and in the claims below. They may be made using the synthetic methods outlined in the Examples section. These methods can be further modified and optimized using the principles and techniques of organic chemistry as applied by a person skilled in the art. Such principles and techniques are taught, for example, in Smith, March's Advanced Organic Chemistry: Reactions, Mechanisms, and Structure, (2013), which is incorporated by reference herein. In addition, the synthetic methods may be further modified and optimized for preparative, pilot- or large-scale production, either batch or continuous, using the principles and techniques of process chemistry as applied by a person skilled in the art. Such principles and techniques are taught, for example, in Anderson, Practical Process Research & Development—A Guide for Organic Chemists (2012), which is incorporated by reference herein.

All the compounds of the present disclosure may in some embodiments be used for the prevention and treatment of one or more diseases or disorders discussed herein or otherwise. In some embodiments, one or more of the compounds characterized or exemplified herein as an intermediate, a metabolite, and/or prodrug, may nevertheless also be useful for the prevention and treatment of one or more diseases or disorders. As such unless explicitly stated to the contrary, all compounds of the present disclosure are deemed “active compounds” and “therapeutic compounds” that are contemplated for use as active pharmaceutical ingredients (APIs). Actual suitability for human or veterinary use is typically determined using a combination of clinical trial protocols and regulatory procedures, such as those administered by the Food and Drug Administration (FDA). In the United States, the FDA is responsible for protecting the public health by assuring the safety, effectiveness, quality, and security of human and veterinary drugs, vaccines and other biological products, and medical devices.

In some embodiments, the compounds of the present disclosure have the advantage that they may be more efficacious than, be less toxic than, be longer acting than, be more potent than, produce fewer side effects than, be more easily absorbed than, more metabolically stable than, more lipophilic than, more hydrophilic than, and/or have a better pharmacokinetic profile (e.g., higher oral bioavailability and/or lower clearance) than, and/or have other useful pharmacological, physical, or chemical properties over, compounds known in the art, whether for use in the indications stated herein or otherwise.

The compounds of the present disclosure may contain one or more asymmetrically-substituted carbon or nitrogen atom and may be isolated in optically active or racemic form. Thus, all chiral, diastereomeric, racemic form, epimeric form, and all geometric isomeric forms of a chemical formula are intended, unless the specific stereochemistry or isomeric form is specifically indicated. Compounds may occur as racemates and racemic mixtures, single enantiomers, diastereomeric mixtures and individual diastereomers. In some embodiments, a single diastereomer is obtained. The chiral centers of the compounds of the present disclosure can have the S or the R configuration. In some embodiments, the present compounds may contain two or more atoms which have a defined stereochemical orientation.

Chemical formulas used to represent compounds of the present disclosure will typically only show one of possibly several different tautomers. For example, many types of ketone groups are known to exist in equilibrium with corresponding enol groups. Similarly, many types of imine groups exist in equilibrium with enamine groups. Regardless of which tautomer is depicted for a given compound, and regardless of which one is most prevalent, all tautomers of a given chemical formula are intended.

In addition, atoms making up the compounds of the present disclosure are intended to include all isotopic forms of such atoms. Isotopes, as used herein, include those atoms having the same atomic number but different mass numbers. By way of general example and without limitation, isotopes of hydrogen include tritium and deuterium, and isotopes of carbon include ¹³C and ¹⁴C.

In some embodiments, compounds of the present disclosure function as prodrugs or can be derivatized to function as prodrugs. Since prodrugs are known to enhance numerous desirable qualities of pharmaceuticals (e.g., solubility, bioavailability, manufacturing, etc.), the compounds employed in some methods of the invention may, if desired, be delivered in prodrug form. Thus, the disclosure contemplates prodrugs of the compounds of the present disclosure as well as methods of delivering prodrugs. Prodrugs of the compounds employed in the disclosure may be prepared by modifying functional groups present in the compound in such a way that the modifications are cleaved, either in routine manipulation or in vivo, to the parent compound. Accordingly, prodrugs include, for example, compounds described herein in which a hydroxy, amino, or carboxy group is bonded to any group that, when the prodrug is administered to a patient, cleaves to form a hydroxy, amino, or carboxylic acid, respectively.

In some embodiments, compounds of the present disclosure exist in salt or non-salt form. With regard to the salt form(s), in some embodiments the particular anion or cation forming a part of any salt form of a compound provided herein is not critical, so long as the salt, as a whole, is pharmacologically acceptable. Additional examples of pharmaceutically acceptable salts and their methods of preparation and use are presented in Handbook of Pharmaceutical Salts: Properties, and Use (2002), which is incorporated herein by reference.

It will be appreciated that many organic compounds can form complexes with solvents in which they are reacted or from which they are precipitated or crystallized. These complexes are known as “solvates.” Where the solvent is water, the complex is known as a “hydrate.” It will also be appreciated that many organic compounds can exist in more than one solid form, including crystalline and amorphous forms. All solid forms of the compounds provided herein, including any solvates thereof are within the scope of the present invention.

### B. Formulations

In another aspect, for administration to a patient in need of such treatment, pharmaceutical formulations (also referred to as a pharmaceutical preparations, pharmaceutical compositions, pharmaceutical products, medicinal products, medicines, medications, or medicaments) comprise a therapeutically effective amount of a compound disclosed herein formulated with one or more excipients and/or drug carriers appropriate to the indicated route of administration. In some embodiments, the compounds disclosed herein are formulated in a manner amenable for the treatment of human and/or veterinary patients. In some embodiments, formulation comprises admixing or combining one or more of the compounds disclosed herein with one or more of the following excipients: lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia, sodium alginate, polyvinylpyrrolidone, and/or polyvinyl alcohol. In some embodiments, e.g., for oral administration, the pharmaceutical formulation may be tableted or encapsulated. In some embodiments, the compounds may be dissolved or slurried in water, polyethylene glycol, propylene glycol, ethanol, corn oil, cottonseed oil, peanut oil, sesame oil, benzyl alcohol, sodium chloride, and/or various buffers. In some embodiments, the pharmaceutical formulations may be subjected to pharmaceutical operations, such as sterilization, and/or may contain drug carriers and/or excipients such as preservatives, stabilizers, wetting agents, emulsifiers, encapsulating agents such as lipids, dendrimers, polymers, proteins such as albumin, nucleic acids, and buffers.

Pharmaceutical formulations may be administered by a variety of methods, e.g., orally or by injection (e.g. subcutaneous, intravenous, and intraperitoneal). Depending on the route of administration, the compounds disclosed herein may be coated in a material to protect the compound from the action of acids and other natural conditions which may inactivate the compound. To administer the active compound by other than parenteral administration, it may be necessary to coat the compound with, or co-administer the compound with, a material to prevent its inactivation. In some embodiments, the active compound may be administered to a patient in an appropriate carrier, for example, liposomes, or a diluent. Pharmaceutically acceptable diluents include saline and aqueous buffer solutions. Liposomes include water-in-oil-in-water CGF emulsions as well as conventional liposomes.

The compounds disclosed herein may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

Pharmaceutical compositions suitable for injectable use include sterile aqueous solutions (where water soluble) or dispersions and sterile powders for the extemporaneous preparation of sterile injectable solutions or dispersion. The carrier can be a solvent or dispersion medium containing, for example, water, ethanol, polyol (such as, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, sodium chloride, or polyalcohols such as mannitol and sorbitol, in the composition. Prolonged absorption of the injectable compositions can be brought about by including in the composition an agent which delays absorption, for example, aluminum monostearate or gelatin.

The compounds disclosed herein can be administered orally, for example, with an inert diluent or an assimilable edible carrier. The compounds and other ingredients may also be enclosed in a hard or soft-shell gelatin capsule, compressed into tablets, or incorporated directly into the patient's diet. For oral therapeutic administration, the compounds disclosed herein may be incorporated with excipients and used in the form of ingestible tablets, buccal tablets, troches, capsules, elixirs, suspensions, syrups, wafers, and the like. The percentage of the therapeutic compound in the compositions and preparations may, of course, be varied. The amount of the therapeutic compound in such pharmaceutical formulations is such that a suitable dosage will be obtained.

The therapeutic compound may also be administered topically to the skin, eye, ear, or mucosal membranes. Administration of the therapeutic compound topically may include formulations of the compounds as a topical solution, lotion, cream, ointment, gel, foam, transdermal patch, or tincture. When the therapeutic compound is formulated for topical administration, the compound may be combined with one or more agents that increase the permeability of the compound through the tissue to which it is administered. In other embodiments, it is contemplated that the topical administration is administered to the eye. Such administration may be applied to the surface of the cornea, conjunctiva, or sclera. Without wishing to be bound by any theory, it is believed that administration to the surface of the eye allows the therapeutic compound to reach the posterior portion of the eye. Ophthalmic topical administration can be formulated as a solution, suspension, ointment, gel, or emulsion. Finally, topical administration may also include administration to the mucosa membranes such as the inside of the mouth. Such administration can be directly to a particular location within the mucosal membrane such as a tooth, a sore, or an ulcer. Alternatively, if local delivery to the lungs is desired the therapeutic compound may be administered by inhalation in a dry-powder or aerosol formulation.

In some embodiments, it may be advantageous to formulate parenteral compositions in dosage unit form for ease of administration and uniformity of dosage. Dosage unit form as used herein refers to physically discrete units suited as unitary dosages for the patients to be treated; each unit containing a predetermined quantity of therapeutic compound calculated to produce the desired therapeutic effect in association with the required pharmaceutical carrier. In some embodiments, the specification for the dosage unit forms of the invention are dictated by and directly dependent on (a) the unique characteristics of the therapeutic compound and the particular therapeutic effect to be achieved, and (b) the limitations inherent in the art of compounding such a therapeutic compound for the treatment of a selected condition in a patient. In some embodiments, active compounds are administered at a therapeutically effective dosage sufficient to treat a condition associated with a condition in a patient. For example, the efficacy of a compound can be evaluated in an animal model system that may be predictive of efficacy in treating the disease in a human or another animal.

In some embodiments, the effective dose range for the therapeutic compound can be extrapolated from effective doses determined in animal studies for a variety of different animals. In some embodiments, the human equivalent dose (HED) in mg/kg can be calculated in accordance with the following formula (see, e.g., Reagan-Shaw et al., FASEB J., 22(3):659-661, 2008, which is incorporated herein by reference):

$$HED ( mg / kg ) = Animal dos e ( mg / kg ) \times ( Animal {K}_{m} / Human {K}_{m} )$$

Use of the Kₘ factors in conversion results in HED values based on body surface area (BSA) rather than only on body mass. Kₘ values for humans and various animals are well known. For example, the Kₘ for an average 60 kg human (with a BSA of 1.6 m²) is 37, whereas a 20 kg child (BSA 0.8 m²) would have a Kₘ of 25. Kₘ for some relevant animal models are also well known, including: mice Kₘ of 3 (given a weight of 0.02 kg and BSA of 0.007); hamster Kₘ of 5 (given a weight of 0.08 kg and BSA of 0.02); rat Kₘ of 6 (given a weight of 0.15 kg and BSA of 0.025) and monkey Kₘ of 12 (given a weight of 3 kg and BSA of 0.24).

Precise amounts of the therapeutic composition depend on the judgment of the practitioner and are specific to each individual. Nonetheless, a calculated HED dose provides a general guide. Other factors affecting the dose include the physical and clinical state of the patient, the route of administration, the intended goal of treatment and the potency, stability and toxicity of the particular therapeutic formulation.

The actual dosage amount of a compound of the present disclosure or composition comprising a compound of the present disclosure administered to a patient may be determined by physical and physiological factors such as type of animal treated, age, sex, body weight, severity of condition, the type of disease being treated, previous or concurrent therapeutic interventions, idiopathy of the patient and on the route of administration. These factors may be determined by a skilled artisan. The practitioner responsible for administration will typically determine the concentration of active ingredient(s) in a composition and appropriate dose(s) for the individual patient. The dosage may be adjusted by the individual physician in the event of any complication.

In some embodiments, the therapeutically effective amount typically will vary from about 0.001 mg/kg to about 1000 mg/kg, from about 0.01 mg/kg to about 750 mg/kg, from about 100 mg/kg to about 500 mg/kg, from about 1 mg/kg to about 250 mg/kg, from about 10 mg/kg to about 150 mg/kg in one or more dose administrations daily, for one or several days (depending of course of the mode of administration and the factors discussed above). Other suitable dose ranges include 1 mg to 10,000 mg per day, 100 mg to 10,000 mg per day, 500 mg to 10,000 mg per day, and 500 mg to 1,000 mg per day. In some embodiments, the amount is less than 10,000 mg per day with a range of 750 mg to 9,000 mg per day.

In some embodiments, the amount of the active compound in the pharmaceutical formulation is from about 2 to about 75 weight percent. In some of these embodiments, the amount if from about 25 to about 60 weight percent.

Single or multiple doses of the agents are contemplated. Desired time intervals for delivery of multiple doses can be determined by one of ordinary skill in the art employing no more than routine experimentation. As an example, patients may be administered two doses daily at approximately 12-hour intervals. In some embodiments, the agent is administered once a day.

The agent(s) may be administered on a routine schedule. As used herein a routine schedule refers to a predetermined designated period of time. The routine schedule may encompass periods of time which are identical, or which differ in length, as long as the schedule is predetermined. For instance, the routine schedule may involve administration twice a day, every day, every two days, every three days, every four days, every five days, every six days, a weekly basis, a monthly basis or any set number of days or weeks there-between. Alternatively, the predetermined routine schedule may involve administration on a twice daily basis for the first week, followed by a daily basis for several months, etc. In other embodiments, the invention provides that the agent(s) may be taken orally and that the timing of which is or is not dependent upon food intake. Thus, for example, the agent can be taken every morning and/or every evening, regardless of when the patient has eaten or will eat.

### II. Methods of Treatment and Combination Therapies

### A. Methods of Treatment

In particular, the compositions that may be used in treating a disease or disorder in a subject (e.g., a human subject) are disclosed herein. The compositions described above are preferably administered to a mammal (e.g., rodent, human, non-human primates, canine, bovine, ovine, equine, feline, etc.) in an effective amount, that is, an amount capable of producing a desirable result in a treated subject (e.g., slowing, stopping, reducing or eliminating one or more symptoms or underlying causes of disease). Toxicity and therapeutic efficacy of the compositions utilized in methods of the disclosure can be determined by standard pharmaceutical procedures. As is well known in the medical and veterinary arts, dosage for any one animal depends on many factors, including the subject's size, body surface area, body weight, age, the particular composition to be administered, time and route of administration, general health, the clinical symptoms and other drugs being administered concurrently. In some embodiments, the amount of the compounds used is calculated to be from about 0.01 mg to about 10,000 mg/day. In some embodiments, the amount is from about 1 mg to about 1,000 mg/day. In some embodiments, the compounds may be administered for 1 day to 20 days. In further embodiments, it is contemplated that the compounds may be administered for 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, or 20 days, or any range derivable therein. In some embodiments, the compounds may be administered for between 3 and 5 days, inclusive. In some embodiments, the compounds may be administered once. It is also contemplated that in some embodiments, the compounds disclosed herein may be administered two or more times. In some embodiments, these dosings may be reduced or increased based upon the biological factors of a particular patient such as increased or decreased metabolic breakdown of the drug or decreased uptake by the digestive tract if administered orally. Additionally, the compounds may be more efficacious and thus a smaller dose is required to achieve a similar effect. Such a dose is typically administered once a day for a few weeks or until sufficient achieve clinical benefit.

The therapeutic methods of the disclosure (which include prophylactic treatment) in general include administration of a therapeutically effective amount of the compositions described herein to a subject in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, family history, and the like).

### B. Combination Therapies

It is envisioned that the compounds described herein may be used in combination therapies with one or more additional therapies or a compound which mitigates one or more of the side effects experienced by the patient. It is common in the field of medicine to combine therapeutic modalities. The following is a general discussion of therapies that may be used in conjunction with the therapies of the present disclosure.

To treat diseases or disorders using the methods and compositions of the present disclosure, one would generally contact a cell or a subject with a compound and at least one other therapy. These therapies would be provided in a combined amount effective to achieve a reduction in one or more disease parameter. This process may involve contacting the cells/subjects with both agents/therapies at the same time, e.g., using a single composition or pharmacological formulation that includes both agents, or by contacting the cell/subject with two distinct compositions or formulations, at the same time, wherein one composition includes the compound and the other includes the other agent. In some embodiments, the compounds of the present disclosure or any therapies used in conjunction with the compounds of the present disclosure may be administered in a less than therapeutically effective dose when used either alone or in combination.

Alternatively, the compounds described herein may precede or follow the other treatment by intervals ranging from minutes to months. Non-limiting examples of such combination therapy include combination of one or more compounds of the invention with another antiviral agent, an anti-inflammatory agent, an immunosuppressant agent, a chemotherapeutic agent, radiation therapy, an antidepressant, an antipsychotic agent, an anticonvulsant, a neutralizing antibody, a mood stabilizer, an anti-infective agent, an antihypertensive agent, a cholesterol-lowering agent or other modulator of blood lipids, an agent for promoting weight loss, an antithrombotic agent, an agent for treating or preventing cardiovascular events such as myocardial infarction or stroke, an antidiabetic agent, an agent for reducing transplant rejection or graft-versus-host disease, an anti-arthritic agent, an analgesic agent, an anti-asthmatic agent or other treatment for respiratory diseases, or an agent for treatment or prevention of skin disorders. Compounds of the invention may be combined with agents designed to improve a patient's immune response to cancer, including (but not limited to) cancer vaccines. See Lu et al. (2011), which is incorporated herein by reference.

It also is conceivable that more than one administration of either the compound or the other therapy will be desired. Various combinations may be employed, where a compound of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Other combinations are also contemplated.

### I. BACTERIA AND BACTERIAL INFECTIONS

In some aspects, the present disclosure provides the TPP containing compounds described herein that may be used to treat a bacterial infection. While humans contain numerous different bacteria on and inside their bodies, an imbalance in bacterial levels or the introduction of pathogenic bacteria can cause a symptomatic bacterial infection. Pathogenic bacteria cause a variety of different diseases including but not limited to numerous foodborne illness, typhoid fever, tuberculosis, pneumonia, syphilis, and leprosy.

Additionally, different bacteria have a wide range of interactions with body and those interactions can modulate ability of the bacteria to cause an infection. For example, bacteria can be conditionally pathogenic such that they only cause an infection under specific conditions. For example, Staphylococcus and Streptococcus bacteria exist in the normal human bacterial biome, but these bacteria when they are allowed to colonize other parts of the body causing a skin infection, pneumonia, or sepsis. Other bacteria are known as opportunistic pathogens and only cause diseases in a patient with a weakened immune system or another disease or disorder.

Bacteria can also be intracellular pathogens which can grow and reproduce within the cells of the host organism. Such bacteria can be divided into two major categories as either obligate intracellular parasites or facultative intracellular parasites. Obligate intracellular parasites require the host cell in order to reproduce and include such bacteria as but are not limited to Chlanydophila, Rickettsia, and Ehrlichia which are known to cause pneumonia, urinary tract infections, typhus, and Rocky Mountain spotted fever. Facultative intracellular parasites can reproduce either intracellular or extracellular. Some non-limiting examples of facultative intracellular parasites include Salmonella, Listeria, Legionella, Mycobacterium, and Brucella which are known to cause food poisoning, typhoid fever, sepsis, meningitis, Legionnaire's disease, tuberculosis, leprosy, and brucellosis.

The TPP containing compounds described herein may be used in the treatment of bacterial infections, including those caused by Staphyloccoccus aureus. S. aureus is a major human pathogen, causing a wide variety of illnesses ranging from mild skin and soft tissue infections and food poisoning to life-threatening illnesses such as deep post-surgical infections, septicaemia, endocarditis, necrotizing pneumonia, and toxic shock syndrome. These organisms have a remarkable ability to accumulate additional antibiotic resistance determinants, resulting in the formation of multiply-drug-resistant strains.

Methicillin, being the first semi-synthetic penicillin to be developed, was introduced in 1959 to overcome the problem of penicillin-resistant S. aureus due to β-lactamase (penicillinase) production (Livermore, 2000). However, methicillin-resistant S. aureus (MRSA) strains were identified soon after the introduction of methicillin (Barber, 1961; Jevons, 1961). The methods described herein may be used in the treatment of MRSA bacterial strains.

Additionally, the antibiotic compounds described herein may be used to treat a Streptococcus pneumoniae infection. Streptococcus pneumoniae is a gram-positive, alpha-hemolytic, bile soluble aerotolerant anaerobe and a member of the genus Streptococcus. A significant human pathogenic bacterium, S. pneumoniae was recognized as a major cause of pneumonia in the late 19th century and is the subject of many humoral immunity studies.

Despite the name, the organism causes many types of pneumococcal infection other than pneumonia, including acute sinusitis, otitis media, meningitis, bacteremia, sepsis, osteomyelitis, septic arthritis, endocarditis, peritonitis, pericarditis, cellulitis, and brain abscess. S. pneumoniae is the most common cause of bacterial meningitis in adults and children and is one of the top two isolates found in ear infection, otitis media. Pneumococcal pneumonia is more common in the very young and the very old.

S. pneumoniae can be differentiated from S. viridans, some of which are also alpha hemolytic, using an optochin test, as S. pneumoniae is optochin sensitive. S. pneumoniae can also be distinguished based on its sensitivity to lysis by bile. The encapsulated, gram-positive coccoid bacteria have a distinctive morphology on gram stain, the so-called, “lancet shape.” It has a polysaccharide capsule that acts as a virulence factor for the organism; more than 90 different serotypes are known, and these types differ in virulence, prevalence, and extent of drug resistance.

S. pneumoniae is part of the normal upper respiratory tract flora but as with many natural flora, it can become pathogenic under the right conditions (e.g., if the immune system of the host is suppressed). Invasins such as Pneumolysin, an anti-phagocytic capsule, various adhesins and immunogenic cell wall components are all major virulence factors.

Finally, bacterial infections could be targeted to a specific location in or on the body. For example, bacteria could be harmless if only exposed to the specific organs, but when it comes in contact with a specific organ or tissue, the bacteria can begin replicating and cause a bacterial infection.

### A. Gram-Positive Bacteria

In some aspects of the present disclosure, the antibiotic compounds described herein may be used to treat a bacterial infection by a gram-positive bacteria. Gram-positive bacteria contain a thick peptidoglycan layer within the cell wall which prevents the bacteria from releasing the stain when dyed with crystal violet. Without being bound by theory, the gram-positive bacteria are often more susceptible to antibiotics. Generally, gram-positive bacteria, in addition to the thick peptidoglycan layer, also comprise a lipid monolayer and contain teichoic acids which react with lipids to form lipoteichoic acids that can act as a chelating agent. Additionally, in gram-positive bacteria, the peptidoglycan layer is outer surface of the bacteria. Many gram-positive bacteria have been known to cause disease including, but are not limited to, Streptococcus, Straphylococcus, Corynebacterium, Enterococcus, Listeria, Bacillus, Clostridium, Rathybacter, Leifsonia, and Clavibacter.

### B. Gram-Negative Bacteria

In some aspects of the present disclosure, the antibiotic compounds described herein may be used to treat a bacterial infection by a gram-negative bacteria. Gram-negative bacteria do not retain the crystal violet stain after washing with alcohol. Gram-negative bacteria, on the other hand, have a thin peptidoglycan layer with an outer membrane of lipopolysaccharides and phospholipids as well as a space between the peptidoglycan and the outer cell membrane called the periplasmic space. Gram-negative bacterial generally do not have teichoic acids or lipoteichoic acids in their outer coating. Generally, gram-negative bacteria also release some endotoxin and contain prions which act as molecular transport units for specific compounds. Most bacteria are gram-negative. Some non-limiting examples of gram-negative bacteria include Bordetella, Borrelia, Burcelia, Campylobacteria, Escherichia, Francisella, Haemophilus, Helicobacter, Legionella, Leptospira, Neisseria, Pseudomonas, Rickettsia, Salmonella, Shigella, Treponema, Vibrio, and Yersinia.

### C. Gram-Indeterminate Bacteria

In some aspects of the present disclosure, the antibiotic compounds herein may be used to treat a bacterial infection by a gram-indeterminate bacteria. Gram-indeterminate bacteria do not full stain or partially stain when exposed to crystal violet. Without being bound by theory, a gram-indeteriminate bacteria may exhibit some of the properties of the gram-positive and gram-negative bacteria. A non-limiting example of a gram-indeterminate bacteria include Mycobacterium tuberculosis or Mycobacterium leprae.

In addition and in elaboration of the examples given above, the term “bacteria” (and derivatives thereof, such as “bacterial infection”) as used in the present disclosure refers to organisms (or infections due to organisms) of the following classes and specific types:
Gram-positive cocci, such as
   Staphylococci (e.g. Staph. aureus, Staph. epidermidis, Staph. saprophyticus, Staph. auricularis, Staph. capitis capitis, Staph. c. ureolyticus, Staph. caprae, Staph. cohnii cohnii, Staph. c. urealyticus, Staph. equorum, Staph. gallinarum, Staph. haemolyticus, Staph. hominis hominis, Staph. h. novobiosepticius, Staph. hyicus, Staph. intermedius, Staph. lugdunensis, Staph. pasteuri, Staph. saccharolyticus, Staph. schleiferi schleiferi, Staph. s. coagulans, Staph. sciuri, Staph. simulans, Staph. warneri and Staph. xylosus) and
   Streptococci (e.g.
      beta-haemolytic, pyogenic streptococci (such as Strept. agalactiae, Strept. canis, Strept. dysgalactiae dysgalactiae, Strept. dysgalactiae equisimilis, Strept. equi equi, Strept. equi zooepidemicus, Strept. iniae, Strept. porcinus and Strept. pyogenes),
      microaerophilic, pyogenic streptococci (Streptococcus “milleri”, such as Strept. anginosus, Strept. constellatus constellatus, Strept. constellatus pharyngidis and Strept. intermedius),
      oral streptococci of the “mitis” (alpha-haemolytic—Streptococcus “viridans”, such as Strept. mitis, Strept. oralis, Strept. sanguinis, Strept. cristatus, Strept. gordonii and Strept. parasanguinis), “salivarius” (non-haemolytic, such as Strept. salivarius and Strept. vestibularis) and “mutans” (tooth-surface streptococci, such as Strept. criceti, Strept. mutans, Strept. ratti and Strept. sobrinus) groups,
      Strept. acidominimus, Strept. bovis, Strept. faecalis, Strept. equinus, Strept. pneumoniae and Strept. suis,
   or Streptococci alternatively classified as Group A, B, C, D, E, G, L, P, U or V Streptococcus);
Gram-negative cocci, such as Neisseria gonorrhoeae, Neisseria meningitidis, Neisseria cinerea, Neisseria elongata, Neisseria flavescens, Neisseria lactamica, Neisseria mucosa, Neisseria sicca, Neisseria subflava and Neisseria weaveri;
Bacillaceae, such as Bacillus anthracis, Bacillus subtilis, Bacillus thuringiensis, Bacillus stearothermophilus and Bacillus cereus;
Enterobacteriaceae, such as
   Escherichia coli,
   Enterobacter (e.g. Enterobacter aerogenes, Enterobacter agglomerans and Enterobacter cloacae)
   Citrobacter (such as Citrob. freundii and Citrob. divernis),
   Hafnia (e.g. Hafnia alvei),
   Erwinia (e.g. Erwinia persicinus),
   Morganella morganii,
   Salmonella (Salmonella enterica and Salmonella typhi),
   Shigella (e.g. Shigella dysenteriae, Shigella flexneri, Shigella boydii and Shigella sonnei),
   Klebsiella (e.g. Klebs. pneumoniae, Klebs. oxytoca, Klebs. ornitholytica, Klebs. planticola, Klebs. ozaenae, Klebs. terrigena, Klebs. granulomatis (Calymmatobacterium granulomatis) and Klebs. rhinoscleromatis),
   Proteus (e.g. Pr. mirabilis, Pr. reugeri and Pr. vulgaris),
   Providencia (e.g. Providencia alcalifaciens, Providencia rettgeri and Providencia stuartii),
   Serratia (e.g. Serratia marcescens and Serratia liquifaciens), and
   Yersinia (e.g. Yersinia enterocolitica, Yersinia pestis and Yersinia pseudotuberculosis);
Enterococci (e.g. Enterococcus avium, Enterococcus casselhflavus, Enterococcus cecorum, Enterococcus dispar, Enterococcus durans, Enterococcus faecalis, Enterococcus faecium, Enterococcus flavescens, Enterococcus gallinarum, Enterococcus hirae, Enterococcus malodoratus, Enterococcus mundtii, Enterococcus pseudoavium, Enterococcus raffinosus and Enterococcus solitarius);
Helicobacter (e.g. Helicobacter pylori, Helicobacter cinaedi and Helicobacter fennelliae);
Acinetobacter (e.g. A. baumanii, A. calcoaceticus, A. haemolyticus, A. johnsonii, A. junii, A. lwoffi and A. radioresistens);
Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri);
Bacteroides fragilis;
Peptococcus (e.g. Peptococcus niger);
Peptostreptococcus;
Clostridium (e.g. C. perfringens, C. difficile, C. botulinum, C. tetani, C. absonum, C. argentinense, C. baratii, C. bifermentans, C. beijerinckii, C. butyricum, C. cadaveris, C. carnis, C. celatum, C. clostridioforme, C. cochlearium, C. cocleatum, C. fallax, C. ghonii, C. glycolicum, C. haemolyticum, C. hastiforme, C. histolyticum, C. indolis, C. innocuum, C. irregulare, C. leptum, C. limosum, C. malenominatum, C. novyi, C. oroticum, C. paraputrificum, C. piliforme, C. putrefasciens, C. ramosum, C. septicum, C. sordelii, C. sphenoides, C. sporogenes, C. subterminale, C. symbiosum and C. tertium);
Mycoplasma (e.g. M. pneumoniae, M. hominis, M. genitalium and M. urealyticum);
Mycobacteria (e.g. Mycobacterium tuberculosis, Mycobacterium avium, Mycobacterium fortuitum, Mycobacterium marinum, Mycobacterium kansasii, Mycobacterium chelonae, Mycobacterium abscessus, Mycobacterium leprae, Mycobacterium smegmitis, Mycobacterium africanum, Mycobacterium alvei, Mycobacterium asiaticum, Mycobacterium aurum, Mycobacterium bohemicum, Mycobacterium bovis, Mycobacterium branderi, Mycobacterium brumae, Mycobacterium celatum, Mycobacterium chubense, Mycobacterium confluentis, Mycobacterium conspicuum, Mycobacterium cookii, Mycobacterium flavescens, Mycobacterium gadium, Mycobacterium gastri, Mycobacterium genavense, Mycobacterium gordonae, Mycobacterium goodii, Mycobacterium haemophilum, Mycobacterium hassicum, Mycobacterium intracellulare, Mycobacterium inteijectum, Mycobacterium heidelberense, Mycobacterium lentiflavum, Mycobacterium malmoense, Mycobacterium microgenicum, Mycobacterium microti, Mycobacterium mucogenicum, Mycobacterium neoaurum, Mycobacterium nonchromogenicum, Mycobacterium peregrinum, Mycobacterium phlei, Mycobacterium scrofulaceum, Mycobacterium shimoidei, Mycobacterium simiae, Mycobacterium szulgai, Mycobacterium terrae, Mycobacterium thermoresistabile, Mycobacterium triplex, Mycobacterium triviale, Mycobacterium tusciae, Mycobacterium ulcerans, Mycobacterium vaccae, Mycobacterium wolinskyi and Mycobacterium xenopi);
Haemophilus (e.g. Haemophilus influenzae, Haemophilus ducreyi, Haemophilus aegyptius, Haemophilus parainfluenzae, Haemophilus haemolyticus and Haemophilus parahaemolyticus);
Actinobacillus (e.g. Actinobacillus actinomycetemcomitans, Actinobacillus equuli, Actinobacillus hominis, Actinobacillus lignieresii, Actinobacillus suis and Actinobacillus ureae);
Actinomyces (e.g. Actinomyces israelii);
Brucella (e.g. Brucella abortus, Brucella canis, Brucella melintensis and Brucella suis);
Campylobacter (e.g. Campylobacter jejuni, Campylobacter coli, Campylobacter lari and Campylobacter fetus);
Listeria monocytogenes;
Vibrio (e.g. Vibrio cholerae and Vibrio parahaemolyticus, Vibrio alginolyticus, Vibrio carchariae, Vibrio fluvialis, Vibrio furnissii, Vibrio hollisae, Vibrio metschnikovii, Vibrio mimicus and Vibrio vulnificus);
Erysipelothrix rhusopathiae;
Corynebacteriaceae (e.g. Corynebacterium diphtheriae, Corynebacterium jeikeum and Corynebacterium urealyticum);
Spirochaetaceae, such as Borrelia (e.g. Borrelia recurrentis, Borrelia burgdorferi, Borrelia afzelii, Borrelia andersonii, Borrelia bissettii, Borrelia garinii, Borrelia japonica, Borrelia lusitaniae, Borrelia tanukii, Borrelia turdi, Borrelia valaisiana, Borrelia caucasica, Borrelia crocidurae, Borrelia duttoni, Borrelia graingeri, Borrelia hermsii, Borrelia hispanica, Borrelia latyschewii, Borrelia mazzottii, Borrelia parkeri, Borrelia persica, Borrelia turicatae and Borrelia venezuelensis) and Treponema (Treponema pallidum ssp. pallidum, Treponema pallidum ssp. endemicum, Treponema pallidum ssp. perienue and Treponema carateum);
Pasteurella (e.g. Pasteurella aerogenes, Pasteurella bettyae, Pasteurella canis, Pasteurella dagmatis, Pasteurella gallinarum, Pasteurella haemolytica, Pasteurella multocida multocida, Pasteurella multocida gallicida, Pasteurella multocida septica, Pasteurella pneumotropica and Pasteurella stomatis);
Bordetella (e.g. Bordetella bronchiseptica, Bordetella hinzii, Bordetella holmseii, Bordetella parapertussis, Bordetella pertussis and Bordetella trematum);
Nocardiaceae, such as Nocardia (e.g. Nocardia asteroides and Nocardia brasiliensis);
Rickeasia (e.g. Ricksettsii or Coxiella burnetii);
Legionella (e.g. Legionalla anisa, Legionalla birminghamensis, Legionalla bozemanii, Legionalla cincinnatiensis, Legionalla dumoffii, Legionalla feeleii, Legionalla gormanii, Legionalla hackeliae, Legionalla israelensis, Legionalla jordanis, Legionalla lansingensis, Legionalla longbeachae, Legionalla maceachernii, Legionalla micdadei, Legionalla oakridgensis, Legionalla pneumophila, Legionalla sainthelensi, Legionalla tucsonensis and Legionalla wadsworthii);
Moraxella catarrhalis;
Stenotrophomonas maltophilia;
Burkholderia cepacia;
Francisella tularensis;
Gardnerella (e.g. Gardneralla vaginalis and Gardneralla mobiluncus);
Streptobacillus moniliformis;
Flavobacteriaceae, such as Capnocytophaga (e.g. Capnocytophaga canimorsus, Capnocytophaga cynodegmi, Capnocytophaga gingivalis, Capnocytophaga granulosa, Capnocytophaga haemolytica, Capnocytophaga ochracea and Capnocytophaga sputigena);
Bartonella (Bartonella bacillformis, Bartonella clarridgeiae, Bartonella elizabethae, Bartonella henselae, Bartonella quintana and Bartonella vinsonii arupensis);
Leptospira (e.g. Leptospira biflexa, Leptospira borgpetersenii, Leptospira inadai, Leptospira interrogans, Leptospira kirschneri, Leptospira noguchii, Leptospira santarosai and Leptospira weilii);
Spirillium (e.g. Spirillum minus);
Bacteroides (e.g. Bacteroides caccae, Bacteroides capillosus, Bacteroides coagulans, Bacteroides distasonis, Bacteroides eggerthii, Bacteroides forsythus, Bacteroides fragilis, Bacteroides merdae, Bacteroides ovatus, Bacteroides putredinis, Bacteroides pyogenes, Bacteroides splanchinicus, Bacteroides stercoris, Bacteroides tectus, Bacteroides thetaiotaomicron, Bacteroides umiformis, Bacteroides ureolyticus and Bacteroides vulgatus);
Prevotella (e.g. Prevotella bivia, Prevotella buccae, Prevotella corporis, Prevotella dentalis (Mitsuokella dentalis), Prevotella denticola, Prevotella disiens, Prevotella enoeca, Prevotella heparinolytica, Prevotella intermedia, Prevotella loeschii, Prevotella melaninogenica, Prevotella nigrescens, Prevotella oralis, Prevotella oris, Prevotella oulora, Prevotella tannerae, Prevotella venoralis and Prevotella zoogleoformans);
Porphyromonas (e.g. Porphyromonas asaccharolytica, Porphyromonas cangingivalis, Porphyromonas canoris, Porphyromonas cansulci, Porphyromonas catoniae, Porphyromonas circumdentaria, Porphyromonas crevioricanis, Porphyromonas endodontalis, Porphyromonas gingivalis, Porphyromonas gingivicanis, Porphyromonas levii and Porphyromonas macacae);
Fusobacterium (e.g. F. gonadiaformans, F. mortiferum, F. naviforme, F. necrogenes, F. necrophorum necrophorum, F. necrophorum fundiliforme, F. nucleatum nucleatum, F. nucleatum fusiforme, F. nucleatum polymorphum, F. nucleatum vincentii, F. periodonticum, F. russii, F. ulcerans and F. varium);
Chlamydia (e.g. Chlamydia trachomatis);
Chlamydophila (e.g. Chlamydophila abortus (Chlamydia psittaci), Chlamydophila pneumoniae (Chlamydia pneumoniae) and Chlamydophila psittaci (Chlamydia psittaci));
Leuconostoc (e.g. Leuconostoc citreum, Leuconostoc cremoris, Leuconostoc dextranicum, Leuconostoc lactis, Leuconostoc mesenteroides and Leuconostoc pseudomesenteroides);
Gemella (e.g. Gemella bergeri, Gemella haemolysans, Gemella morbillorum and Gemella sanguinis); and
Ureaplasma (e.g. Ureaplasma parvum and Ureaplasma urealyticum).

Thus, compounds of the invention may be used to kill any of the above-mentioned bacterial organisms.

In particular, the present compounds may be used to treat infections of the following bacteria.

Bacillaceae, such as Bacillus anthracis, Bacillus subtilis, Bacillus thuringiensis, Bacillus stearothermophilus and Bacillus cereus;

Staphylococci, such as Staph. aureus (either Methicillin-sensitive (i.e. MSSA) or Methicillin-resistant (i.e. MRSA)) and Staph. epidermidis;
Acinetobacter (e.g. A. baumanii, A. calcoaceticus, A. haemolyticus, A. johnsonii, A. junii, A. lwoffi and A. radioresistens);
Enterobacteriaceae, such as Escherichia coli, Klebsiella (e.g. Klebs. pneumoniae and Klebs. oxytoca) and Proteus (e.g. Pr. mirabilis, Pr. rettgeri and Pr. vulgaris); or
Pseudomonas (e.g. Ps. aeruginosa, Ps. maltophilia (Stenotrophomonas maltophilia), Ps. alcaligenes, Ps. chlororaphis, Ps. fluorescens, Ps. luteola. Ps. mendocina, Ps. monteilii, Ps. oryzihabitans, Ps. pertocinogena, Ps. pseudalcaligenes, Ps. putida and Ps. stutzeri).

The compounds of the present invention are particularly advantageous as they may be capable of inhibiting the growth, survival and reproduction of Gram negative bacteria, something which few existing antibacterial agents are able to do effectively. Thus, in particular embodiments of all of the methods disclosed herein, the bacteria are Gram negative bacteria.

In this respect, particular conditions that the compounds and formulations of the invention can be used to treat include tuberculosis (e.g. pulmonary tuberculosis, non-pulmonary tuberculosis (such as tuberculosis lymph glands, genito-urinary tuberculosis, tuberculosis of bone and joints, tuberculosis meningitis) and miliary tuberculosis), anthrax, abscesses, acne vulgaris, actinomycosis, bacilliary dysentry, bacterial conjunctivitis, bacterial keratitis, botulism, Buruli ulcer, bone and joint infections, bronchitis (acute or chronic), brucellosis, burn wounds, cat scratch fever, cellulitis, chancroid, cholangitis, cholecystitis, cutaneous diphtheria, cystic fibrosis, cystitis, diffuse panbronchiolitis, diphtheria, dental caries, diseases of the upper respiratory tract, empymea, endocarditis, endometritis, enteric fever, enteritis, epididymitis, epiglottitis, erysipelas, erysipeloid, erythrasma, eye infections, furuncles, Gardnerella vaginitis, gastrointestinal infections (gastroenteritis), genital infections, gingivitis, gonorrhoea, granuloma inguinale, Haverhill fever, infected burns, infections following dental operations, infections in the oral region, infections associated with prostheses, intraabdominal abscesses, Legionnaire's disease, leprosy, leptospirosis, listeriosis, liver abscesses, Lyme disease, lymphogranuloma venerium, mastitis, mastoiditis, meningitis and infections of the nervous system, mycetoma, nocardiosis (e.g. Madura foot), non-specific urethritis, opthalmia (e.g. opthalmia neonatorum), osteomyelitis, otitis (e.g. otitis externa and otitis media), orchitis, pancreatitis, paronychia, pelveoperitonitis, peritonitis, peritonitis with appendicitis, pharyngitis, phlegmons, pinta, plague, pleural effusion, pneumonia, postoperative wound infections, postoperative gas gangrene, prostatitis, pseudo-membranous colitis, psittacosis, pulmonary emphysema, pyelonephritis, pyoderma (e.g. impetigo), Q fever, rat-bite fever, reticulosis, Ritter's disease, salmonellosis, salpingitis, septic arthritis, septic infections, septicameia, sinusitis, skin infections (e.g. skin granulomas), syphilis, systemic infections, tonsillitis, toxic shock syndrome, trachoma, tularaemia, typhoid, typhus (e.g. epidemic typhus, murine typhus, scrub typhus and spotted fever), urethritis, wound infections, yaws, aspergillosis, candidiasis (e.g. oropharyngeal candidiasis, vaginal candidiasis or balanitis), cryptococcosis, favus, histoplasmosis, intertrigo, mucormycosis, tinea (e.g. tinea corporis, tinea capitis, tinea cruris, tinea pedis and tinea unguium), onychomycosis, pityriasis versicolor, ringworm and sporotrichosis.

Further conditions that may be mentioned in this respect include infections with MSSA, MRSA, Staph. epidermidis, Strept. agalactiae, Strept. pyogenes, Escherichia coli, Klebs. pneumoniae, Klebs. oxytoca, Pr. mirabilis, Pr. rettgeri, Pr. vulgaris, Haemophilis influenzae, Enterococcus faecalis or Enterococcus faecium.

The compounds and formulations of the invention will normally be administered orally, subcutaneously, intravenously, intraarterially, transdermally, intranasally, by inhalation, or by any other parenteral route, in the form of pharmaceutical preparations comprising the active ingredient either as a free base or a non-toxic organic or inorganic acid addition salt, in a pharmaceutically acceptable dosage form. Depending upon the disorder and patient to be treated, as well as the route of administration, the compounds and formulations may be administered at varying doses.

Additionally, compositions of the invention may have the advantage that they may be more efficacious than, be less toxic than, have a broader range of activity than, be more potent than, produce fewer side effects than, or have other useful pharmacological properties over compositions known in the prior art. In particular, compositions of the invention may have the advantage that they are less toxic than compositions known in the prior art due to a reduction in the detrimental effects that the delivery agents may have on cell membrane (of the host organisms).

### III. ANTIBIOTICS

The term “antibiotics” are drugs that may be used to treat a bacterial infection through either inhibiting the growth of bacteria or killing bacteria. Without being bound by theory, it is believed that antibiotics can be classified into two major classes: bactericidal agents that kill bacteria or bacteriostatic agents that slow down or prevent the growth of bacteria.

The first commercially available antibiotic was released in the 1930s. Since then, many different antibiotics have been developed and widely prescribed. In 2010, on average, 4 in 5 Americans were prescribed antibiotics annually. Given the prevalence of antibiotics, bacteria have started to develop resistance to specific antibiotics and antibiotic mechanisms. Without being bound by theory, the use of antibiotics in combination with another antibiotic, may modulate resistance and enhance the efficacy of one or both agents. The antibiotics, either alone or in combination, may optionally be delivered in the form of a prodrug as described and defined elsewhere herein.

### A. Ribosome Inhibitors and the Limitations to Kill Gram Negative Pathogens

Pleuromutilin was first discovered in the basidiomycete Pleurotus mutilus (Kavanagh et al., 1951) and is an inhibitor of ribosomal protein synthesis. The pleuromutilins inhibit protein synthesis by binding to the central part of domain V of the 50S ribosomal subunit at the peptidyl transferase center. This prevents the correct positioning of the CCA ends of the tRNAs for the peptide transfer in the A- and P-site thus inhibiting peptide bond formation (Hagenauer, 1975; Hagenauer & Ruf, 1981). Derivatives such as tiamulin and valnemulin are two well established derivatives in veterinary medicine for oral and intramuscular administration. Lefamulin, a semi-synthetic pleuromutilin compound highly active against multi-resistant pathogens, is a promising antibiotic recently approved by the US FDA for the treatment of community-acquired bacterial pneumonia (McCarthy, 2021). Pleuromutilins typically have potent activity against gram-positive, some gram-negative pathogens, and are unaffected by resistance compared to other major antibiotic classes such as macrolides, fluoroquinolones, tetracyclines, beta-lactam antibiotics, and others which is attributed to its unique and highly specific mode of action (Paukner & Riedl, 2017; Eyal et al., 2016). However, known ribosomal resistance mechanisms have been reported (Long et al., 2006; Gentry et al., 2007). Lefamulin notably lacks activity against most anaerobes as well as Pseudomonas aeruginosa, Acinetobacter baumannii, and E. faecalis (McCarthy, 2021). However, it was suggested that pleuromutilins should possess antibacterial activity against these strains since coupled in vitro transcription-translation assay results showed inhibition of the bacterial translation in these organisms (Paukner & Riedl, 2017). This suggests that the drug has poor exposure in these gram-negative pathogens either due to poor cell permeability or because the drug is rapidly effluxed out of the cell before it can elicit its antibiotic effect. Research at Nabriva revealed that the intrinsic resistance of Enterobacteriaceae is caused by the efflux of pleuromutilins mediated by the AcrAB-TolC efflux pump (Paukner & Riedl, 2017). This is supported by the fact that AcrABToIC deficient E. coli strains were susceptible to pleuromutilins (Paukner & Riedl, 2017) and that the minimum inhibitory concentration (MIC) values against Enterobacteriaceae were significantly reduced by the addition of efflux pump inhibitors (e.g., PAPN). Therefore, pleuromutilin seemed like an excellent antibiotic to use to study the feasibility of a pleuromutilin-TPP⁺ conjugate to compare side-by-side with pleuromutilin as a means to overcome its limitations.

In some embodiments, the compounds of the present disclosure may comprise antibiotics which may belong to a different class than pleuromutilins. The present disclosure also contemplates the use of presently disclosed compounds in combination therapy, as discussed above, with at least a second antibiotic. In some embodiments, the compounds of the present disclosure may comprise multiple antibiotic moieties, including but not limited to a pleuromutilin.

In any of the above embodiments, the antibiotics contemplated for compounds of the present disclosure may fall into a wide range of classes. In some embodiments, the antibiotic may be a narrow spectrum antibiotic which targets a specific bacteria type. In some embodiments, the antibiotic may be a bactericidal antibiotic. Non-limiting examples of bactericidal antibiotics include penicillin, cephalosporin, polymyxin, rifamycin, lipiarmycin, quinolones, and sulfonamides. In some embodiments, the antibiotic may be bacteriostatic antibiotic. Non-limiting examples of bacteriostatic antibiotics include macrolides, lincosamides, or tetracyclines. In some embodiments, the antibiotic is an aminoglycoside such as kanamycin and streptomycin, an ansamycin such as rifaximin and geldanamycin, a carbacephem such as loracarbef, a carbapenem such as ertapenem, imipenem, a cephalosporin such as cephalexin, cefixime, cefepime, and ceftobiprole, a glycopeptide such as vancomycin or teicoplanin, a lincosamide such as lincomycin and clindamycin, a lipopeptide such as daptomycin, a macrolide such as clarithromycin, spiramycin, azithromycin, and telithromycin, a monobactam such as aztreonam, a nitrofuran such as furazolidone and nitrofurantoin, an oxazolidonones such as linezolid, a penicillin such as amoxicillin, azlocillin, flucloxacillin, and penicillin G, an antibiotic polypeptide such as bacitracin, polymyxin B, and colistin, a quinolone such as ciprofloxacin, levofloxacin, and gatifloxacin, a sulfonamide such as silver sulfadiazine, mefenide, sulfadimethoxine, or sulfasalazine, or a tetracycline such as demeclocycline, doxycycline, minocycline, oxytetracycline, or tetracycline. In some embodiments, the compounds could comprise a drug that acts against mycobacteria such as cycloserine, capreomycin, ethionamide, rifampicin, rifabutin, rifapentine, and streptomycin. The present disclosure contemplates compounds that comprise or administered in combination therapies with other antibiotics that may include arsphenamine, chloramphenicol, fosfomycin, fusidic acid, metronidazole, mupirocin, platensimycin, quinupristin, dalfopristin, thiamphenicol, tigecycline, tinidazole, or trimethoprim.

The compounds of the invention are useful because they possess pharmacological activity. They are therefore indicated as pharmaceuticals.

The use of compounds or compositions used herein includes their use as pharmaceuticals (both for human and veterinary use). The compositions of the present invention may also be useful in other fields of industry. For example, the compositions may be useful as plant protection products (i.e. in agriculture), in cosmetic products (e.g. in creams, toothpaste, lotions and ointments), and hygiene and sterilization procedures (e.g. in scientific laboratories).

In some aspects, the present disclosure may be used as:
(a) a compound or composition of the invention, as hereinbefore defined, for use in treating or preventing a bacterial infection in a subject;
(b) use of a compound or composition of the invention, as hereinbefore defined, in the manufacture of a medicament for treating or preventing a bacterial infection in a subject;
(c) a method of treating or preventing a bacterial infection, which method comprises administration of a therapeutically effective amount of a compound or composition of the invention as hereinbefore defined to a subject in need thereof;
(d) use (e.g. in vitro or ex vivo use) of a compound or composition of the invention to kill bacteria.

### IV. CHEMISTRY BACKGROUND

In some aspects, compounds of this disclosure can be synthesized using the methods of organic chemistry as described in this application. These methods can be further modified and optimized using the principles and techniques of organic chemistry as applied by a person skilled in the art. Such principles and techniques are taught, for example, in March's Advanced Organic Chemistry: Reactions, Mechanisms, and Structure (2007), which is incorporated by reference herein.

The synthetic methods described herein can be further modified and optimized for preparative, pilot- or large-scale production, either batch of continuous, using the principles and techniques of process chemistry as applied by a person skilled in the art. Such principles and techniques are taught, for example, in Practical Process Research & Development (2000), which is incorporated by reference herein. The synthetic method described herein may be used to produce preparative scale amounts of the compounds described herein.

### A. Chemical Definitions

When used in the context of a chemical group: “hydrogen” means —H; “hydroxy” means —OH; “oxo” means ═O; “carbonyl” means —C(═O)—; “carboxy” means —C(═O)OH (also written as —COOH or —CO₂H); “halo” means independently —F, —Cl, —Br or —I; “amino” means —NH₂; “hydroxyamino” means —NHOH; “nitro” means —NO₂; imino means=NH; “cyano” means —CN; “isocyanyl” means —N═C═O; “azido” means —N₃; in a monovalent context “phosphate” means —OP(O)(OH)₂ or a deprotonated form thereof; in a divalent context “phosphate” means OP(O)(OH)O— or a deprotonated form thereof; “mercapto” means —SH; and “thio” means ═S; “thiocarbonyl” means —C(═S)—; “sulfonyl” means —S(O)₂—; and “sulfinyl” means —S(O)—.

In the context of chemical formulas, the symbol “ [symbol] ” means a single bond, “ [symbol] ” means a double bond, and “ [symbol] ” means triple bond. The symbol “ [symbol] ” represents an optional bond, which if present is either single or double. The symbol “ [symbol] ” represents a single bond or a double bond. Thus, the formula

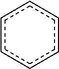

covers, for example

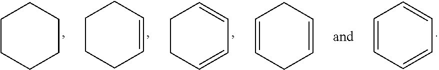

And it is understood that no one such ring atom forms p Tre than one double bond. Furthermore, it is noted that the covalent bond symbol “ [symbol] ”, when connecting one or two stereogenic atoms, does not indicate any preferred stereochemistry. Instead, it covers all stereoisomers as well as mixtures thereof. The symbol “ [symbol] ”, when drawn perpendicularly across a bond (e.g.

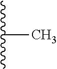

for methyl) indicates a point of attachment of the group. It is noted that the point of attachment is typically only identified in this manner for larger groups in order to assist the reader in unambiguously identifying a point of attachment. The symbol “ [symbol] ” means a single bond where the group attached to the thick end of the wedge is “out of the page.” The symbol “ [symbol] ” means a single bond where the group attached to the thick end of the wedge is “into the page”. The symbol “ [symbol] ” means a single bond where the geometry around a double bond (e.g., either E or Z) is undefined. Both options, as well as combinations thereof are therefore intended. Any undefined valency on an atom of a structure shown in this application implicitly represents a hydrogen atom bonded to that atom. A bold dot on a carbon atom indicates that the hydrogen attached to that carbon is oriented out of the plane of the paper.

When a variable is depicted as a “floating group” on a ring system, for example, the group “R” in the formula:

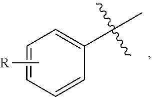

then the variable may replace any hydrogen atom attached to any of the ring atoms, including a depicted, implied, or expressly defined hydrogen, so long as a stable structure is formed. When a variable is depicted as a “floating group” on a fused ring system, as for example the group “R” in the formula:

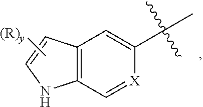

then the variable may replace any hydrogen attached to any of the ring atoms of either of the fused rings unless specified otherwise. Replaceable hydrogens include depicted hydrogens (e.g., the hydrogen attached to the nitrogen in the formula above), implied hydrogens (e.g., a hydrogen of the formula above that is not shown but understood to be present), expressly defined hydrogens, and optional hydrogens whose presence depends on the identity of a ring atom (e.g., a hydrogen attached to group X, when X equals —CH—), so long as a stable structure is formed. In the example depicted, R may reside on either the 5-membered or the 6-membered ring of the fused ring system. In the formula above, the subscript letter “y” immediately following the R enclosed in parentheses, represents a numeric variable. Unless specified otherwise, this variable can be 0, 1, 2, or any integer greater than 2, only limited by the maximum number of replaceable hydrogen atoms of the ring or ring system.

For the chemical groups and compound classes, the number of carbon atoms in the group or class is as indicated as follows: “Cn” or “C=n” defines the exact number (n) of carbon atoms in the group/class. “C≤n” defines the maximum number (n) of carbon atoms that can be in the group/class, with the minimum number as small as possible for the group/class in question. For example, it is understood that the minimum number of carbon atoms in the groups “alkyl_{(C≤8)}”, “alkanediyl_{(C≤8)}”, “heteroaryl_{(C≤8)}”, and “acyl_{(C≤8)}” is one, the minimum number of carbon atoms in the groups “alkenyl_{(C≤8)}”, “alkynyl_{(C≤8)}”, and “heterocycloalkyl_{(C≤8)}” is two, the minimum number of carbon atoms in the group “cycloalkyl_{(C≤8)}” is three, and the minimum number of carbon atoms in the groups “aryl_{(C≤8)}” and “arenediyl_{(C≤8)}” is six. “Cn-n′” defines both the minimum (n) and maximum number (n′) of carbon atoms in the group. Thus, “alkyl_{(C2-10)}” designates those alkyl groups having from 2 to 10 carbon atoms. These carbon number indicators may precede or follow the chemical groups or class it modifies and it may or may not be enclosed in parenthesis, without signifying any change in meaning. Thus, the terms “C₁₋₄-alkyl”, “C1-4-alkyl”, “alkyl_{(C1-4)}”, and “alkyl_{(C≤4)}” are all synonymous. Except as noted below, every carbon atom is counted to determine whether the group or compound falls with the specified number of carbon atoms. For example, the group dihexylamino is an example of a dialkylamino_{(C12)} group; however, it is not an example of a dialkylamino_{(C6)} group. Likewise, phenylethyl is an example of an aralkyl_{(C=8)} group. When any of the chemical groups or compound classes defined herein is modified by the term “substituted”, any carbon atom in the moiety replacing the hydrogen atom is not counted. Thus methoxyhexyl, which has a total of seven carbon atoms, is an example of a substituted alkyl_{(C1-6)}. Unless specified otherwise, any chemical group or compound class listed in a claim set without a carbon atom limit has a carbon atom limit of less than or equal to twelve.

The term “saturated” when used to modify a compound or chemical group means the compound or chemical group has no carbon-carbon double and no carbon-carbon triple bonds, except as noted below. When the term is used to modify an atom, it means that the atom is not part of any double or triple bond. In the case of substituted versions of saturated groups, one or more carbon oxygen double bond or a carbon nitrogen double bond may be present. And when such a bond is present, then carbon-carbon double bonds that may occur as part of keto-enol tautomerism or imine/enamine tautomerism are not precluded. When the term “saturated” is used to modify a solution of a substance, it means that no more of that substance can dissolve in that solution.

The term “aliphatic” signifies that the compound or chemical group so modified is an acyclic or cyclic, but non-aromatic compound or group. In aliphatic compounds/groups, the carbon atoms can be joined together in straight chains, branched chains, or non-aromatic rings (alicyclic). Aliphatic compounds/groups can be saturated, that is joined by single carbon-carbon bonds (alkanes/alkyl), or unsaturated, with one or more carbon-carbon double bonds (alkenes/alkenyl) or with one or more carbon-carbon triple bonds (alkynes/alkynyl).

The term “aromatic” signifies that the compound or chemical group so modified has a planar unsaturated ring of atoms with 4n+2 electrons in a fully conjugated cyclic π system. An aromatic compound or chemical group may be depicted as a single resonance structure; however, depiction of one resonance structure is taken to also refer to any other resonance structure. For example:

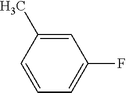

is also taken to refer to

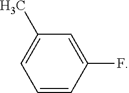

Aromatic compounds may also be depicted using a circle to represent the delocalized nature of the electrons in the fully conjugated cyclic E system, two non-limiting examples of which are shown below:

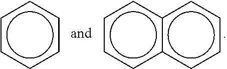

The term “alkyl” refers to a monovalent saturated aliphatic group with a carbon atom as the point of attachment, a linear or branched acyclic structure, and no atoms other than carbon and hydrogen. The groups —CH₃ (Me), —CH₂CH₃ (Et), —CH₂CH₂CH₃ (n-Pr or propyl), —CH(CH₃)₂ (i-Pr, ⁱPr or isopropyl), —CH₂CH₂CH₂CH₃ (n-Bu), —CH(CH₃)CH₂CH₃ (sec-butyl), —CH₂CH(CH₃)₂ (isobutyl), —C(CH₃)₃ (tert-butyl, t-butyl, t-Bu or ^{t}Bu), and —CH₂C(CH₃)₃ (neo-pentyl) are non-limiting examples of alkyl groups. The term “alkanediyl” refers to a divalent saturated aliphatic group, with one or two saturated carbon atom(s) as the point(s) of attachment, a linear or branched acyclic structure, no carbon-carbon double or triple bonds, and no atoms other than carbon and hydrogen. The groups —CH₂— (methylene), —CH₂CH₂—, —CH₂C(CH₃)₂CH₂—, and —CH₂CH₂CH₂— are non-limiting examples of alkanediyl groups. The term “alkylidene” refers to the divalent group ═CRR′ in which R and R′ are independently hydrogen or alkyl. Non-limiting examples of alkylidene groups include: ═CH₂, ═CH(CH₂CH₃), and ═C(CH₃)₂. An “alkane” refers to the class of compounds having the formula H—R, wherein R is alkyl as this term is defined above.

The term “cycloalkyl” refers to a monovalent saturated aliphatic group with a carbon atom as the point of attachment, said carbon atom forming part of one or more non-aromatic ring structures, no carbon-carbon double or triple bonds, and no atoms other than carbon and hydrogen. Non-limiting examples include: —CH(CH₂)₂ (cyclopropyl), cyclobutyl, cyclopentyl, or cyclohexyl (Cy). As used herein, the term does not preclude the presence of one or more alkyl groups (carbon number limitation permitting) attached to a carbon atom of the non-aromatic ring structure. The term “cycloalkanediyl” refers to a divalent saturated aliphatic group with two carbon atoms as points of attachment, no carbon-carbon double or triple bonds, and no atoms other than carbon and hydrogen. The group

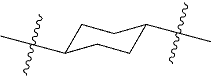 [SMILES: *C1CCC(*)CC1]

is a non-limiting example of cycloalkanediyl group. A “cycloalkane” refers to the class of compounds having the formula H—R, wherein R is cycloalkyl as this term is defined above.

The term “alkenyl” refers to a monovalent unsaturated aliphatic group with a carbon atom as the point of attachment, a linear or branched, acyclic structure, at least one nonaromatic carbon-carbon double bond, no carbon-carbon triple bonds, and no atoms other than carbon and hydrogen. Non-limiting examples include: —CH═CH₂ (vinyl), —CH═CHCH₃, —CH═CHCH₂CH₃, —CH₂CH═CH₂ (allyl), —CH₂CH═CHCH₃, and —CH═CHCH═CH₂. The term “alkenediyl” refers to a divalent unsaturated aliphatic group, with two carbon atoms as points of attachment, a linear or branched acyclic structure, at least one nonaromatic carbon-carbon double bond, no carbon-carbon triple bonds, and no atoms other than carbon and hydrogen. The groups —CH═CH—, —CH═C(CH₃)CH₂—, —CH═CHCH₂—, and —CH₂CH═CHCH₂— are non-limiting examples of alkenediyl groups. It is noted that while the alkenediyl group is aliphatic, once connected at both ends, this group is not precluded from forming part of an aromatic structure. The terms “alkene” and “olefin” are synonymous and refer to the class of compounds having the formula H—R, wherein R is alkenyl as this term is defined above. Similarly, the terms “terminal alkene” and “α-olefin” are synonymous and refer to an alkene having just one carbon-carbon double bond, wherein that bond is part of a vinyl group at an end of the molecule.

The term “alkynyl” refers to a monovalent unsaturated aliphatic group with a carbon atom as the point of attachment, a linear or branched acyclic structure, at least one carbon-carbon triple bond, and no atoms other than carbon and hydrogen. As used herein, the term alkynyl does not preclude the presence of one or more non-aromatic carbon-carbon double bonds. The groups —C≡CH, —C≡CCH₃, and —CH₂C≡CCH₃ are non-limiting examples of alkynyl groups. An “alkyne” refers to the class of compounds having the formula H—R, wherein R is alkynyl.

The term “aryl” refers to a monovalent unsaturated aromatic group with an aromatic carbon atom as the point of attachment, said carbon atom forming part of a one or more aromatic ring structures, each with six ring atoms that are all carbon, and wherein the group consists of no atoms other than carbon and hydrogen. If more than one ring is present, the rings may be fused or unfused. Unfused rings are connected with a covalent bond. As used herein, the term aryl does not preclude the presence of one or more alkyl groups (carbon number limitation permitting) attached to the first aromatic ring or any additional aromatic ring present. Non-limiting examples of aryl groups include phenyl (Ph), methylphenyl, (dimethyl)phenyl, —C₆H₄CH₂CH₃ (ethylphenyl), naphthyl, and a monovalent group derived from biphenyl (e.g., 4-phenylphenyl). The term “arenediyl” refers to a divalent aromatic group with two aromatic carbon atoms as points of attachment, said carbon atoms forming part of one or more six-membered aromatic ring structures, each with six ring atoms that are all carbon, and wherein the divalent group consists of no atoms other than carbon and hydrogen. As used herein, the term arenediyl does not preclude the presence of one or more alkyl groups (carbon number limitation permitting) attached to the first aromatic ring or any additional aromatic ring present. If more than one ring is present, the rings may be fused or unfused. Unfused rings are connected with a covalent bond. Non-limiting examples of arenediyl groups include:

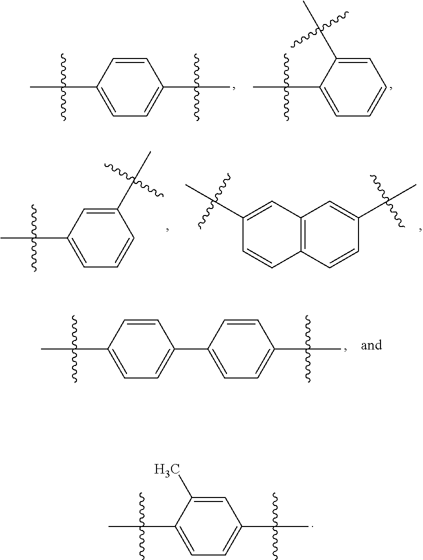

An “arene” refers to the class of compounds having the formula H—R, wherein R is aryl as that term is defined above. Benzene and toluene are non-limiting examples of arenes.

The term “aralkyl” refers to the monovalent group -alkanediyl-aryl, in which the terms alkanediyl and aryl are each used in a manner consistent with the definitions provided above. Non-limiting examples are: phenylmethyl (benzyl, Bn) and 2-phenyl-ethyl.

The term “heteroaryl” refers to a monovalent aromatic group with an aromatic carbon atom or nitrogen atom as the point of attachment, said carbon atom or nitrogen atom forming part of one or more aromatic ring structures, each with three to eight ring atoms, wherein at least one of the ring atoms of the aromatic ring structure(s) is nitrogen, oxygen or sulfur, and wherein the heteroaryl group consists of no atoms other than carbon, hydrogen, aromatic nitrogen, aromatic oxygen and aromatic sulfur. If more than one ring is present, the rings are fused; however, the term heteroaryl does not preclude the presence of one or more alkyl or aryl groups (carbon number limitation permitting) attached to one or more ring atoms. Non-limiting examples of heteroaryl groups include benzoxazolyl, benzimidazolyl, furanyl, imidazolyl (Im), indolyl, indazolyl, isoxazolyl, methylpyridinyl, oxazolyl, oxadiazolyl, phenylpyridinyl, pyridinyl (pyridyl), pyrrolyl, pyrimidinyl, pyrazinyl, quinolyl, quinazolyl, quinoxalinyl, triazinyl, tetrazolyl, thiazolyl, thienyl, and triazolyl. The term “N-heteroaryl” refers to a heteroaryl group with a nitrogen atom as the point of attachment. A “heteroarene” refers to the class of compounds having the formula H—R, wherein R is heteroaryl. Pyridine and quinoline are non-limiting examples of heteroarenes.

The term “heteroaralkyl” refers to the monovalent group -alkanediyl-heteroaryl, in which the terms alkanediyl and heteroaryl are each used in a manner consistent with the definitions provided above. Non-limiting examples are: pyridinylmethyl and 2-quinolinyl-ethyl.

The term “heterocycloalkyl” refers to a monovalent non-aromatic group with a carbon atom or nitrogen atom as the point of attachment, said carbon atom or nitrogen atom forming part of one or more non-aromatic ring structures, each with three to eight ring atoms, wherein at least one of the ring atoms of the non-aromatic ring structure(s) is nitrogen, oxygen or sulfur, and wherein the heterocycloalkyl group consists of no atoms other than carbon, hydrogen, nitrogen, oxygen and sulfur. If more than one ring is present, the rings are fused. As used herein, the term does not preclude the presence of one or more alkyl groups (carbon number limitation permitting) attached to one or more ring atoms. Also, the term does not preclude the presence of one or more double bonds in the ring or ring system, provided that the resulting group remains non-aromatic. Non-limiting examples of heterocycloalkyl groups include aziridinyl, azetidinyl, pyrrolidinyl, piperidinyl, piperazinyl, morpholinyl, thiomorpholinyl, tetrahydrofuranyl, tetrahydrothiofuranyl, tetrahydropyranyl, pyranyl, oxiranyl, and oxetanyl. The term “N-heterocycloalkyl” refers to a heterocycloalkyl group with a nitrogen atom as the point of attachment. N-pyrrolidinyl is an example of such a group.

The term “heterocycloalkalkyl” refers to the monovalent group-alkanediyl-heterocycloalkyl, in which the terms alkanediyl and heterocycloalkyl are each used in a manner consistent with the definitions provided above. Non-limiting examples are: morpholinylmethyl and piperidinylethyl.

The term “acyl” refers to the group —C(O)R, in which R is a hydrogen, alkyl, cycloalkyl, or aryl as those terms are defined above. The groups, —CHO, —C(O)CH₃ (acetyl, Ac), —C(O)CH₂CH₃, —C(O)CH(CH₃)₂, —C(O)CH(CH₂)₂, —C(O)C₆H₅, and —C(O)C₆H₄CH₃ are non-limiting examples of acyl groups. A “thioacyl” is defined in an analogous manner, except that the oxygen atom of the group —C(O)R has been replaced with a sulfur atom, —C(S)R. The term “aldehyde” corresponds to an alkyl group, as defined above, attached to a —CHO group.

The term “alkoxy” refers to the group —OR, in which R is an alkyl, as that term is defined above. Non-limiting examples include: —OCH₃ (methoxy), —OCH₂CH₃ (ethoxy), —OCH₂CH₂CH₃, —OCH(CH₃)₂ (isopropoxy), or —OC(CH₃)₃ (tert-butoxy). The terms “cycloalkoxy”, “alkenyloxy”, “alkynyloxy”, “aryloxy”, “aralkoxy”, “heteroaryloxy”, “heterocycloalkoxy”, and “acyloxy”, when used without the “substituted” modifier, refers to groups, defined as —OR, in which R is cycloalkyl, alkenyl, alkynyl, aryl, aralkyl, heteroaryl, heterocycloalkyl, and acyl, respectively. The term “alkylthio” and “acylthio” refers to the group —SR, in which R is an alkyl and acyl, respectively. The term “alcohol” corresponds to an alkane, as defined above, wherein at least one of the hydrogen atoms has been replaced with a hydroxy group. The term “ether” corresponds to an alkane, as defined above, wherein at least one of the hydrogen atoms has been replaced with an alkoxy group.

The term “alkylamino” refers to the group —NHR, in which R is an alkyl, as that term is defined above. Non-limiting examples include: —NHCH₃ and —NHCH₂CH₃. The term “dialkylamino” refers to the group —NRR′, in which R and R′ can be the same or different alkyl groups. Non-limiting examples of dialkylamino groups include: —N(CH₃)₂ and —N(CH₃)(CH₂CH₃). The term “amido” (acylamino), when used without the “substituted” modifier, refers to the group —NHR, in which R is acyl, as that term is defined above. A non-limiting example of an amido group is —NHC(O)CH₃.

When a chemical group is used with the “substituted” modifier, one or more hydrogen atom has been replaced, independently at each instance, by —OH, —F, —Cl, —Br, —I, —NH₂, —NO₂, —CO₂H, —CO₂CH₃, —CO₂CH₂CH₃, —CN, —SH, —OCH₃, —OCH₂CH₃, —C(O)CH₃, —NHCH₃, —NHCH₂CH₃, —N(CH₃)₂, —C(O)NH₂, —C(O)NHCH₃, —C(O)N(CH₃)₂, —OC(O)CH₃, —NHC(O)CH₃, —S(O)₂OH, or —S(O)₂NH₂. For example, the following groups are non-limiting examples of substituted alkyl groups: —CH₂OH, —CH₂Cl, —CF₃, —CH₂CN, —CH₂C(O)OH, —CH₂C(O)OCH₃, —CH₂C(O)NH₂, —CH₂C(O)CH₃, —CH₂OCH₃, —CH₂OC(O)CH₃, —CH₂NH₂, —CH₂N(CH₃)₂, and —CH₂CH₂Cl. The term “haloalkyl” is a subset of substituted alkyl, in which the hydrogen atom replacement is limited to halo (i.e. —F, —Cl, —Br, or —I) such that no other atoms aside from carbon, hydrogen and halogen are present. The group, —CH₂Cl is a non-limiting example of a haloalkyl. The term “fluoroalkyl” is a subset of substituted alkyl, in which the hydrogen atom replacement is limited to fluoro such that no other atoms aside from carbon, hydrogen and fluorine are present. The groups —CH₂F, —CF₃, and —CH₂CF₃ are non-limiting examples of fluoroalkyl groups. The term “haloaryl” is a subset of substituted aryl, in which the hydrogen atom replacement is limited to halo (i.e. —F, —Cl, —Br, or —I) such that no other atoms aside from carbon, hydrogen and halogen are present. The group, —C₆H₄Cl is a non-limiting example of a haloaryl group. Non-limiting examples of substituted aralkyls are: (3-chlorophenyl)-methyl, and 2-chloro-2-phenyl-eth-1-yl. The groups, —C(O)CH₂CF₃, —CO₂H (carboxyl), —CO₂CH₃ (methylcarboxyl), —CO₂CH₂CH₃, —C(O)NH₂ (carbamoyl), and —CON(CH₃)₂, are non-limiting examples of substituted acyl groups. The groups —NHC(O)OCH₃ and —NHC(O)NHCH₃ are non-limiting examples of substituted amido groups.

The use of the word “a” or “an,” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the inherent variation of error for the device, the method being employed to determine the value, or the variation that exists among the study subjects or patients. Unless otherwise noted, the term “about” is used to indicate a value of ±10% of the reported value, preferably a value of ±5% of the reported value. It is to be understood that, whenever the term “about” is used, a specific reference to the exact numerical value indicated is also included.”

An “active ingredient” (AI) or active pharmaceutical ingredient (API) (also referred to as an active compound, active substance, active agent, pharmaceutical agent, agent, biologically active molecule, or a therapeutic compound) is the ingredient in a pharmaceutical drug that is biologically active.

An “amine protecting group” or “amino protecting group” is well understood in the art. An amine protecting group is a group which modulates the reactivity of the amine group during a reaction which modifies some other portion of the molecule. Amine protecting groups can be found at least in Greene and Wuts, 1999, which is incorporated herein by reference. Some non-limiting examples of amino protecting groups include formyl, acetyl, propionyl, pivaloyl, t-butylacetyl, 2-chloroacetyl, 2-bromoacetyl, trifluoroacetyl, trichloroacetyl, o-nitrophenoxyacetyl, α-chlorobutyryl, benzoyl, 4-chlorobenzoyl, 4-bromobenzoyl, 4-nitrobenzoyl, and the like; sulfonyl groups such as benzenesulfonyl, p-toluenesulfonyl and the like; alkoxy- or aryloxycarbonyl groups (which form urethanes with the protected amine) such as benzyloxycarbonyl (Cbz), p-chlorobenzyloxycarbonyl, p-methoxybenzyloxycarbonyl, p-nitrobenzyloxycarbonyl, 2-nitrobenzyloxycarbonyl, p-bromobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 2-nitro-4,5-dimethoxybenzyloxycarbonyl, 3,4,5-trimethoxybenzyloxycarbonyl, 1-(p-biphenylyl)-1-methylethoxycarbonyl, α,α-dimethyl-3,5-dimethoxybenzyloxycarbonyl, benzhydryloxycarbonyl, t-butyloxycarbonyl (Boc), diisopropylmethoxycarbonyl, isopropyloxycarbonyl, ethoxycarbonyl, methoxycarbonyl, allyloxycarbonyl (Alloc), 2,2,2-trichloroethoxycarbonyl, 2-trimethyl-silylethyloxycarbonyl (Teoc), phenoxycarbonyl, 4-nitrophenoxycarbonyl, fluorenyl-9-methoxycarbonyl (Fmoc), cyclopentyloxycarbonyl, adamantyloxycarbonyl, cyclohexyl-oxycarbonyl, phenylthiocarbonyl and the like; alkylaminocarbonyl groups (which form ureas with the protect amine) such as ethylaminocarbonyl and the like; aralkyl groups such as benzyl, triphenylmethyl, benzyloxymethyl and the like; and silyl groups such as trimethylsilyl and the like. Additionally, the “amine protecting group” can be a divalent protecting group such that both hydrogen atoms on a primary amine are replaced with a single protecting group. In such a situation the amine protecting group can be phthalimide (phth) or a substituted derivative thereof wherein the term “substituted” is as defined above. In some embodiments, the halogenated phthalimide derivative may be tetrachlorophthalimide (TCphth). When used herein, a “protected amino group”, is a group of the formula PG_{MA}NH— or PG_{DA}N— wherein PG_{MA} is a monovalent amine protecting group, which may also be described as a “monovalently protected amino group” and PG_{DA} is a divalent amine protecting group as described above, which may also be described as a “divalently protected amino group”.

The term “canonical amino acid” refers to any one of alanine, arginine, asparagine, aspartate (or aspartic acid), cysteine, glutamine, glutamate (or glutamic acid), glycine, histidine, isoleucine, leucine, lysine, methionine, phenylalanine, proline, serine, threonine, tryptophan, tyrosine, or valine. The “side chain” of the amino acid refers to any atom or group(s) of atoms attached to the α-carbon of the amino acid molecule.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and also covers other unlisted steps.

The term “effective,” as that term is used in the specification and/or claims, means adequate to accomplish a desired, expected, or intended result. “Effective amount,” “Therapeutically effective amount” or “pharmaceutically effective amount” when used in the context of treating a patient or subject with a compound means that amount of the compound which, when administered to the patient or subject, is sufficient to effect such treatment or prevention of the disease as those terms are defined below.

An “excipient” is a pharmaceutically acceptable substance formulated along with the active ingredient(s) of a medication, pharmaceutical composition, formulation, or drug delivery system. Excipients may be used, for example, to stabilize the composition, to bulk up the composition (thus often referred to as “bulking agents,” “fillers,” or “diluents” when used for this purpose), or to confer a therapeutic enhancement on the active ingredient in the final dosage form, such as facilitating drug absorption, reducing viscosity, or enhancing solubility. Excipients include pharmaceutically acceptable versions of antiadherents, binders, coatings, colors, disintegrants, flavors, glidants, lubricants, preservatives, sorbents, sweeteners, and vehicles. The main excipient that serves as a medium for conveying the active ingredient is usually called the vehicle. Excipients may also be used in the manufacturing process, for example, to aid in the handling of the active substance, such as by facilitating powder flowability or non-stick properties, in addition to aiding in vitro stability such as prevention of denaturation or aggregation over the expected shelf life. The suitability of an excipient will typically vary depending on the route of administration, the dosage form, the active ingredient, as well as other factors.

The term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

As used herein, the term “IC₅₀” refers to an inhibitory dose which is 50% of the maximum response obtained. This quantitative measure indicates how much of a particular drug or other substance (inhibitor) is needed to inhibit a given biological, biochemical or chemical process (or component of a process, i.e. an enzyme, cell, cell receptor or microorganism) by half. The term “EC₅₀” refers to an amount that is an effective concentration to results in a half-maximal response.

An “isomer” of a first compound is a separate compound in which each molecule contains the same constituent atoms as the first compound, but where the configuration of those atoms in three dimensions differs.

As used herein, the term “patient” or “subject” refers to a living mammalian organism, such as a human, monkey, cow, sheep, goat, dog, cat, mouse, rat, guinea pig, or transgenic species thereof. In certain embodiments, the patient or subject is a primate. Non-limiting examples of human patients are adults, juveniles, infants and fetuses.

As generally used herein “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

“Pharmaceutically acceptable salts” means salts of compounds disclosed herein which are pharmaceutically acceptable, as defined above, and which possess the desired pharmacological activity. Such salts include acid addition salts formed with inorganic acids such as hydrochloric acid, hydrobromic acid, sulfuric acid, nitric acid, phosphoric acid, and the like; or with organic acids such as 1,2-ethanedisulfonic acid, 2-hydroxyethanesulfonic acid, 2-naphthalenesulfonic acid, 3-phenylpropionic acid, 4,4′-methylenebis(3-hydroxy-2-ene-1-carboxylic acid), 4-methylbicyclo[2.2.2]oct-2-ene-1-carboxylic acid, acetic acid, aliphatic mono- and dicarboxylic acids, aliphatic sulfuric acids, aromatic sulfuric acids, benzenesulfonic acid, benzoic acid, camphorsulfonic acid, carbonic acid, cinnamic acid, citric acid, cyclopentanepropionic acid, ethanesulfonic acid, fumaric acid, glucoheptonic acid, gluconic acid, glutamic acid, glycolic acid, heptanoic acid, hexanoic acid, hydroxynaphthoic acid, lactic acid, laurylsulfuric acid, maleic acid, malic acid, malonic acid, mandelic acid, methanesulfonic acid, muconic acid, o-(4-hydroxybenzoyl)benzoic acid, oxalic acid, p-chlorobenzenesulfonic acid, phenyl-substituted alkanoic acids, propionic acid, p-toluenesulfonic acid, pyruvic acid, salicylic acid, stearic acid, succinic acid, tartaric acid, tertiarybutylacetic acid, trimethylacetic acid, and the like. Pharmaceutically acceptable salts also include base addition salts which may be formed when acidic protons present are capable of reacting with inorganic or organic bases. Acceptable inorganic bases include sodium hydroxide, sodium carbonate, potassium hydroxide, aluminum hydroxide and calcium hydroxide. Acceptable organic bases include ethanolamine, diethanolamine, triethanolamine, tromethamine, N-methylglucamine and the like. It should be recognized that the particular anion or cation forming a part of any salt of this invention is not critical, so long as the salt, as a whole, is pharmacologically acceptable. Additional examples of pharmaceutically acceptable salts and their methods of preparation and use are presented in Handbook of Pharmaceutical Salts: Properties, and Use (P. H. Stahl & C. G. Wermuth eds., Verlag Helvetica Chimica Acta, 2002).

A “pharmaceutically acceptable carrier,” “drug carrier,” or simply “carrier” is a pharmaceutically acceptable substance formulated along with the active ingredient medication that is involved in carrying, delivering and/or transporting a chemical agent. Drug carriers may be used to improve the delivery and the effectiveness of drugs, including for example, controlled-release technology to modulate drug bioavailability, decrease drug metabolism, and/or reduce drug toxicity. Some drug carriers may increase the effectiveness of drug delivery to the specific target sites. Examples of carriers include: liposomes, microspheres (e.g., made of poly(lactic-co-glycolic) acid), albumin microspheres, synthetic polymers, nanofibers, protein-DNA complexes, protein conjugates, erythrocytes, virosomes, and dendrimers.

A “pharmaceutical drug” (also referred to as a pharmaceutical, pharmaceutical preparation, pharmaceutical composition, pharmaceutical formulation, pharmaceutical product, medicinal product, medicine, medication, medicament, or simply a drug, agent, or preparation) is a composition used to diagnose, cure, treat, or prevent disease, which comprises an active pharmaceutical ingredient (API) (defined above) and optionally contains one or more inactive ingredients, which are also referred to as excipients (defined above).

“Prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

“Prodrug” or “pro-drug” means a compound that is convertible in vivo metabolically into an active pharmaceutical ingredient of the present invention. The prodrug itself may or may not have activity with its prodrug form. For example, a compound comprising a hydroxy group may be administered as an ester that is converted by hydrolysis in vivo to the hydroxy compound. Non-limiting examples of suitable esters that may be converted in vivo into hydroxy compounds include acetates, citrates, lactates, phosphates, tartrates, malonates, oxalates, salicylates, propionates, succinates, fumarates, maleates, methylene-bis-β-hydroxynaphthoate, gentisates, isethionates, di-p-toluoyltartrates, methanesulfonates, ethanesulfonates, benzenesulfonates, p-toluenesulfonates, cyclohexylsulfamates, quinates, and esters of amino acids. Similarly, a compound comprising an amine group may be administered as an amide that is converted by hydrolysis in vivo to the amine compound.

A “stereoisomer” or “optical isomer” is an isomer of a given compound in which the same atoms are bonded to the same other atoms, but where the configuration of those atoms in three dimensions differs. “Enantiomers” are stereoisomers of a given compound that are mirror images of each other, like left and right hands. “Diastereomers” are stereoisomers of a given compound that are not enantiomers. Chiral molecules contain a chiral center, also referred to as a stereocenter or stereogenic center, which is any point, though not necessarily an atom, in a molecule bearing groups such that an interchanging of any two groups leads to a stereoisomer. In organic compounds, the chiral center is typically a carbon, phosphorus or sulfur atom, though it is also possible for other atoms to be stereocenters in organic and inorganic compounds. A molecule can have multiple stereocenters, giving it many stereoisomers. In compounds whose stereoisomerism is due to tetrahedral stereogenic centers (e.g., tetrahedral carbon), the total number of hypothetically possible stereoisomers will not exceed 2ⁿ, where n is the number of tetrahedral stereocenters. Molecules with symmetry frequently have fewer than the maximum possible number of stereoisomers. A 50:50 mixture of enantiomers is referred to as a racemic mixture. Alternatively, a mixture of enantiomers can be enantiomerically enriched so that one enantiomer is present in an amount greater than 50%. Typically, enantiomers and/or diastereomers can be resolved or separated using techniques known in the art. It is contemplated that that for any stereocenter or axis of chirality for which stereochemistry has not been defined, that stereocenter or axis of chirality can be present in its R form, S form, or as a mixture of the R and S forms, including racemic and non-racemic mixtures. As used herein, the phrase “substantially free from other stereoisomers” means that the composition contains ≤15%, more preferably ≤10%, even more preferably ≤5%, or most preferably ≤1% of another stereoisomer(s).

“Treatment” or “treating” includes (1) inhibiting a disease in a subject or patient experiencing or displaying the pathology or symptomatology of the disease (e.g., arresting further development of the pathology and/or symptomatology), (2) ameliorating a disease in a subject or patient that is experiencing or displaying the pathology or symptomatology of the disease (e.g., reversing the pathology and/or symptomatology), and/or (3) effecting any measurable decrease in a disease or symptom thereof in a subject or patient that is experiencing or displaying the pathology or symptomatology of the disease.

The term “unit dose” refers to a formulation of the compound or composition such that the formulation is prepared in a manner sufficient to provide a single therapeutically effective dose of the active ingredient to a patient in a single administration. Such unit dose formulations that may be used include but are not limited to a single tablet, capsule, or other oral formulations, or a single vial with a syringeable liquid or other injectable formulations.

The above definitions supersede any conflicting definition in any reference that is incorporated by reference herein. The fact that certain terms are defined, however, should not be considered as indicative that any term that is undefined is indefinite. Rather, all terms used are believed to describe the invention in terms such that one of ordinary skill can appreciate the scope and practice the present invention.

### V. EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1—Synthesis of a Pleuromutilin-TPP⁺ Conjugate

Pleuromutilin is commercially available (CAS #125-65-5) and was used directly for derivatization into a triphenylphosponium ester pro-drug. An ester linkage was selected for use to conjugate pleuromutilin with [(4-carboxyphenyl)methyl]triphenyl-phosphonium bromide (CAS #81638-08-6) since ultimately the TPP⁺ moiety was desired to be metabolically released from the inhibitor once the conjugate is delivered to the bacteria. Therefore, triphenylphosphine was reacted with 4-(bromomethyl)benzoic acid in acetone at elevated temperature for 4 hours to produce [(4-carboxyphenyl)methyl]triphenyl-phosphonium bromide. Then this acid was used to esterify pleuromutilin by reaction with 1-ethyl-3-(3-dimethylaminopropyl)carbodiimide (EDCI) in DMF (Scheme 1).

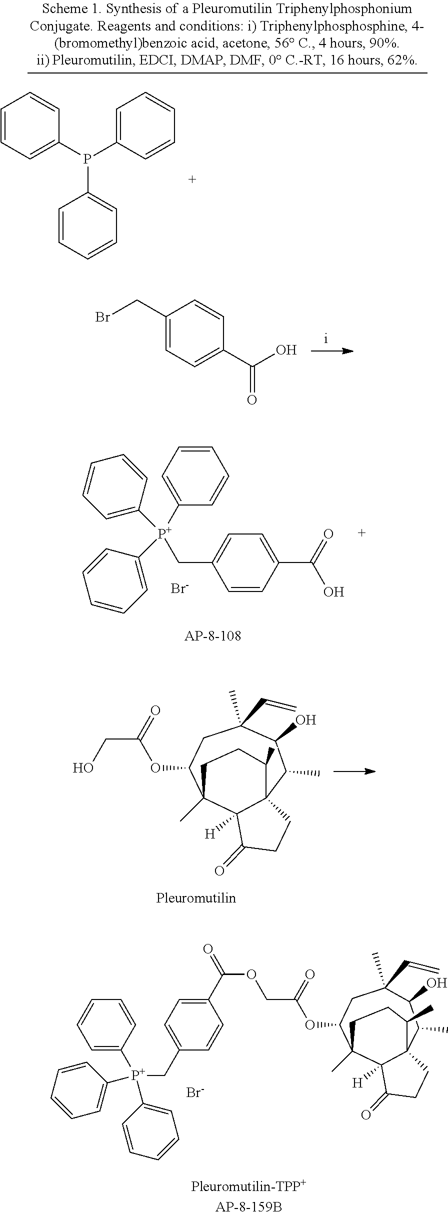

### Example 2—Comparison of Pleuromutilin and Pleuromutilin-TPP⁺ in Model Systems of Gram-Positive and Gram-Negative Bacteria

To compare the efficacy of pleuromutilin and the pleuromulin-TPP⁺ ester pro-drug (AP-8-159B) gram-negative and gram-positive pathogen model system were used. Thus, Escherichia coli (BL21(DE3) from New England Biolabs) was used as a model strain for gram-negative bacteria and clinical isolates of Staphylococcus aureus were used as the model system for gram-positive bacteria (NR-49120, NR-46413 obtained from BEI Resources). All strains were cultured at 37° C. overnight in Luria Bertani (LB) (BD 244610). For testing the drug sensitivity, bacteria were grown in RPMI medium containing 10% fetal bovine serum or human serum.

A resazurin blue assay (alamarBlue®) (Toté et al., 2009; Costa et al., 2021; Wannigama et al., 2019; Travnickova et al., 2019) was used to evaluate bacterial cell viability. Resazurin (7-hydroxy-10-oxidophenoxazin-10-ium-3-one, sodium) is a blue fluorogenic dye used as a redox indicator in cell viability and proliferation assays for bacteria, yeast, or mammalian cells. The resazurin assay protocol is based on the reduction of oxidized non-fluorescent blue resazurin to a red fluorescent dye (resorufin), where NADPH dehydrogenase, without being bound by theory, is probably responsible for the transference of electrons from NADPH+H⁺ to resazurin (Costa et al., 2021). The amount of resorufin produced is directly proportional to the number of living cells. This simple fluorometric-based assay rapidly quantifies metabolically active bacterial cells. Two controls were added to the plate to confirm the correct response from the resazurin. The positive control used evaluated media alone without bacterial culture to confirm there was no change from blue to red. The negative control evaluated the bacteria culture alone without added compound to provide a 100% growth control (red). Thus, resazurin sodium salt (Sigma R7017) was added to pleuromutilin or pleuromutilin-TPP+ prodrug treated bacterial samples at a final concentration of 0.02% and incubated for 8 hours at 37° C. Changes in fluorescence were measured on the Biotek Synergy 2 plate reader after an 8-hour incubation at 37° C. using an excitation filter range of 530-570 nm and an emission filter range of 590-620 nm. An increase in fluorescence intensity corresponded to bacterial growth and was quantified by comparison with untreated bacterial control samples. The ratio of (T_{Threshold} (untreated)/T_{Threshold} (prodrug-treated)) (where T=time) was used to quantify the change in bacterial growth. To minimize inter-experimental variations, T_{Threshold} values were corrected by subtracting untreated control cultures to reach minimum detectable fluorescence. At the end of the experiment, wells were visualized for changes in color from blue (inviable bacteria) to red (viable bacteria) and a picture was taken.

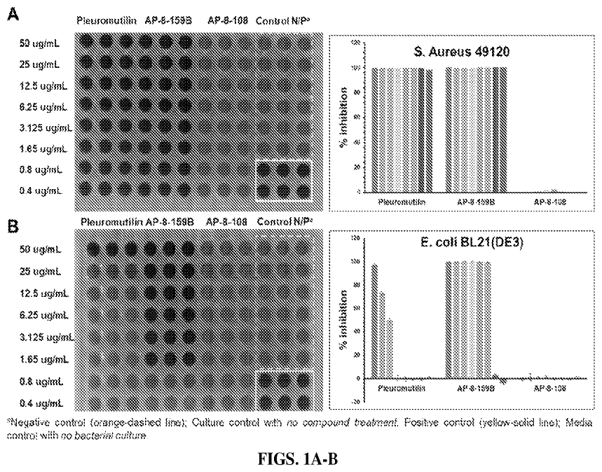

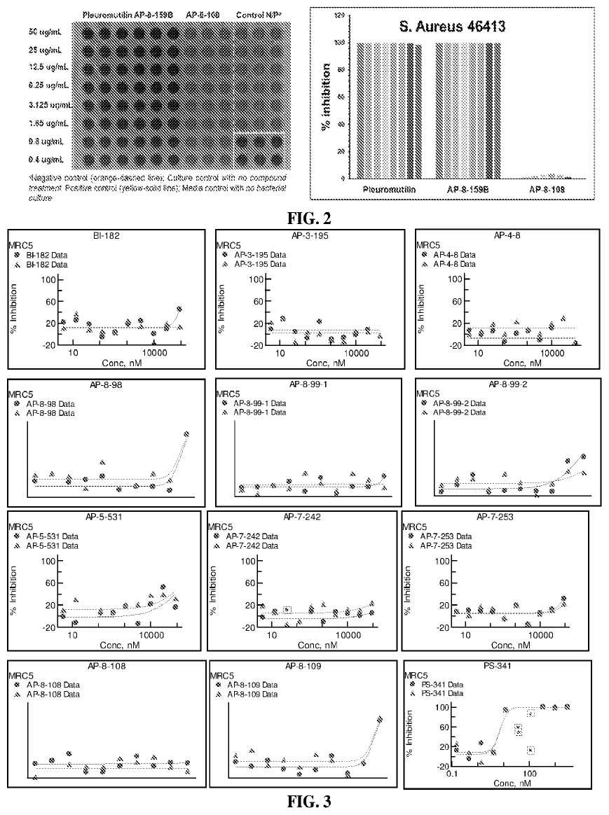

Interestingly, both pleuromutilin and pleuromutilin-TPP⁺ are potent cytotoxic agents to both clinical strains of Staphylococcus aureus that we tested as expected (FIG. 1A and FIG. 2) (Gentry et al., 2007). Pleuromutilin was much less cytotoxic against the model Gram-negative bacteria E Coli BL21 (DE3) used (Jeong et al., 2015). However, the pleuromutilin-TPP⁺ pro-drug showed an increase in cytotoxicity potency from 25 ug/mL for pleuromutilin to 1.65 ug/mL for pleuromutilin-TPP⁺. To rule out toxicity of the TPP⁺ linker alone, AP-8-108 (Scheme 1) was tested side-by side and shown to have no cytotoxic effect on either the Staphylococcus aureus or E. coli BL21 (DE3) strains.

### Example 3—Experimental Procedures

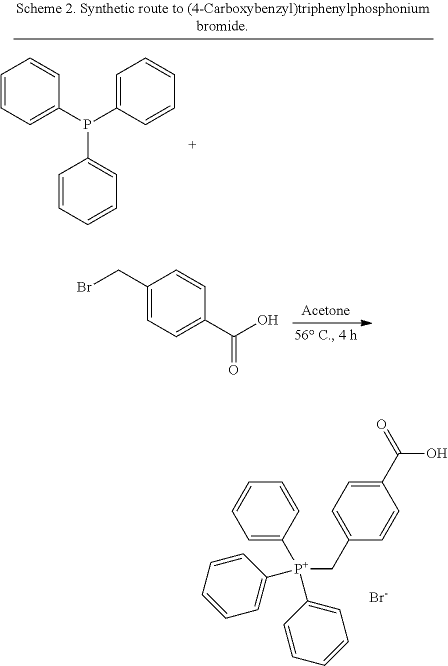 [SMILES: c1ccc(P(c2ccccc2)c2ccccc2)cc1 ; O=C(O)c1ccc(CBr)cc1 ; O=C(O)c1ccc(C[P+](c2ccccc2)(c2ccccc2)c2ccccc2)cc1 ; [Br-]]

(4-Carboxybenzyl)triphenylphosphonium bromide: A stirred solution of 4-(bromomethyl)benzoic acid (5.75 g, 26.74 mmol) and triphenylphosphine (7.05 g, 26.74 mmol) in 125 ml dry acetone was heated to 56° C. for 4 hours. Completion of the reaction was confirmed by LC-MS. Then the solvent was evaporated under reduced pressure and 500 mL of diethyl ether was added to the residue. The resulting white solid precipitate was filtered and dried under high vacuum to provide the product (11.5 g, 24.09 mmol, 90%). The desired product was confirmed by ¹H NMR and LC-MS. ¹H NMR (400 MHz, DMSO) δ 13.10 (s, 1H), 7.99-7.87 (m, 3H), 7.82-7.65 (m, 14H), 7.10 (dd, J=8.4, 2.3 Hz, 2H), 5.29 (d, J=16.3 Hz, 2H). LC-MS m/z: found 397.32, [C₂₆H₂₂O₂P]⁺, [M−Br]⁺, calcd mass 397. 14.

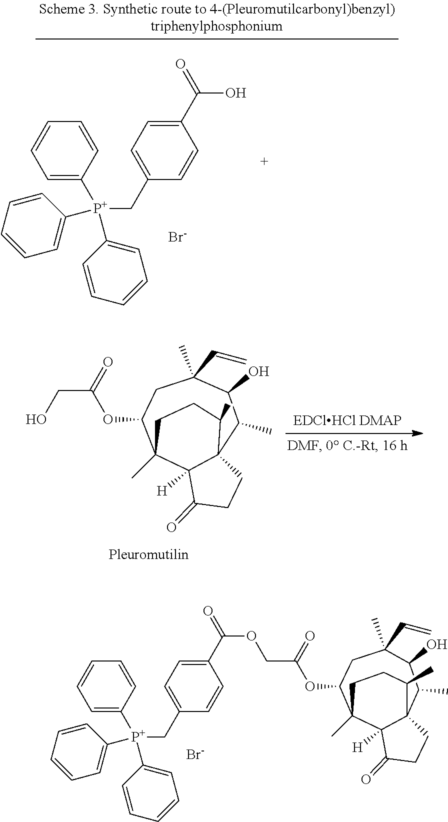 [SMILES: O=C(O)c1ccc(C[P+](c2ccccc2)(c2ccccc2)c2ccccc2)cc1 ; [Br-] ; C=C[C@]1(C)C[C@@H](OC(=O)CO)[C@]2(C)CC[C@@H](C)[C@@]3(CCC(=O)[C@@H]23)[C@@H](C)[C@@H]1O ; C=C[C@]1(C)C[C@@H](OC(=O)COC(=O)c2ccc(C[P+](c3ccccc3)(c3ccccc3)c3ccccc3)cc2)[C@]2(C)CC[C@@H](C)[C@@]3(CCC(=O)[C@@H]23)[C@@H](C)[C@@H]1O ; [Br-]]

4-(Pleuromutilcarbonyl)benzyl)triphenylphosphonium: To a stirred solution of (4-carboxybenzyl)triphenylphosphonium bromide (144 mg, 0.26 mmol) and pleuromutilin (109 mg, 0.29 mmol) in 4 mL of dry DMF at 0° C. was added N,N-dimethylpyridin-4-amine (38 mg, 0.31 mmol) and 1-Ethyl-3-(3-dimethylaminopropyl)carbodiimide (91 mg 0.52 mmol). The reaction mixture was stirred and slowly allowed to warm to room temp and stirred for 16 hours. Completion of reaction was confirmed by LC-MS, then the reaction mixture was diluted with 20 mL of cold water and the product was extracted with ethyl acetate (3×10 mL). The combined organic layer was washed with 10 mL of 10% aq HCl, 10 mL saturated aq NaHCO₃ and 10 mL of brine solution and dried over anhydrous Na₂SO₄. The solvent was evaporated under reduced pressure to provide crude product which was purified by column chromatography (CH₂Cl₂/MeOH) to afford the product as white solid (135 mg, 0.161 mmol, 62%). Product confirmed by ¹H NMR And LC-MS. ¹H NMR (400 MHz, CDCl3) δ 7.79 (ddd, J=18.1, 8.4, 7.0 Hz, 11H), 7.63 (td, J=7.8, 3.5 Hz, 5H), 7.33-7.12 (m, 3H), 6.47 (dd, J=17.4, 11.0 Hz, 1H), 5.97-5.66 (m, 3H), 5.41-5.27 (m, 2H), 5.22 (dd, J=17.4, 1.2 Hz, 1H), 4.72 (dd, J=44.4, 16.0 Hz, 2H), 3.37 (dd, J=10.1, 6.5 Hz, 1H), 2.24 (ddt, J=19.4, 12.3, 8.0 Hz, 3H), 2.13-1.99 (m, 2H), 1.83-1.61 (m, 4H), 1.58-1.32 (m, 7H), 1.30-1.05 (m, 5H), 1.17 (d, J=8.2 Hz, 3H), 0.87 (d, J=7.0 Hz, 3H), 0.77 (d, J=7.0 Hz, 3H). LC-MS m/z: found 757.38, [C₄₈H₅₄O₆P]⁺, [M−Br]⁺, calcd mass 757.37.

Similarly, a TPP⁺-derivative of Lefamulin was planned to be prepared as described in Proposed Scheme 1.

Proposed Scheme 1: Synthesis of Lefamulin-TPP⁺ Derivative

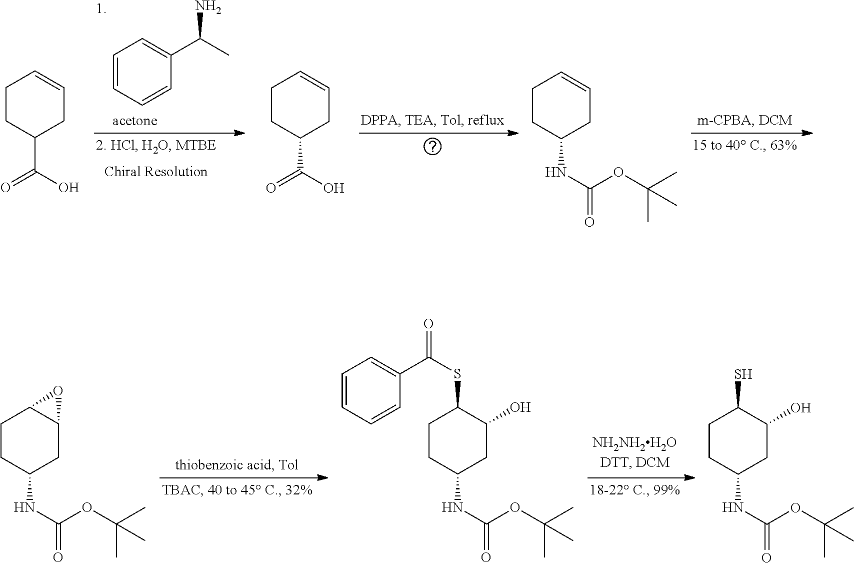

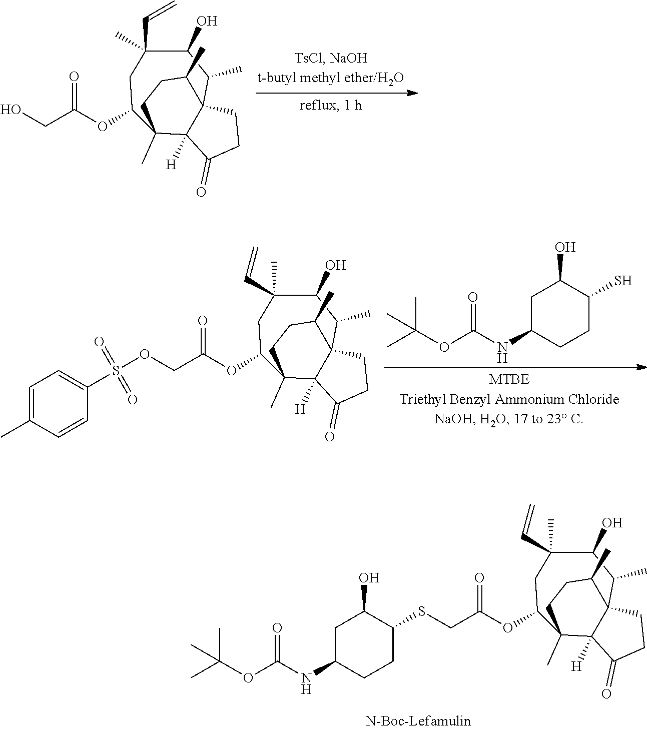

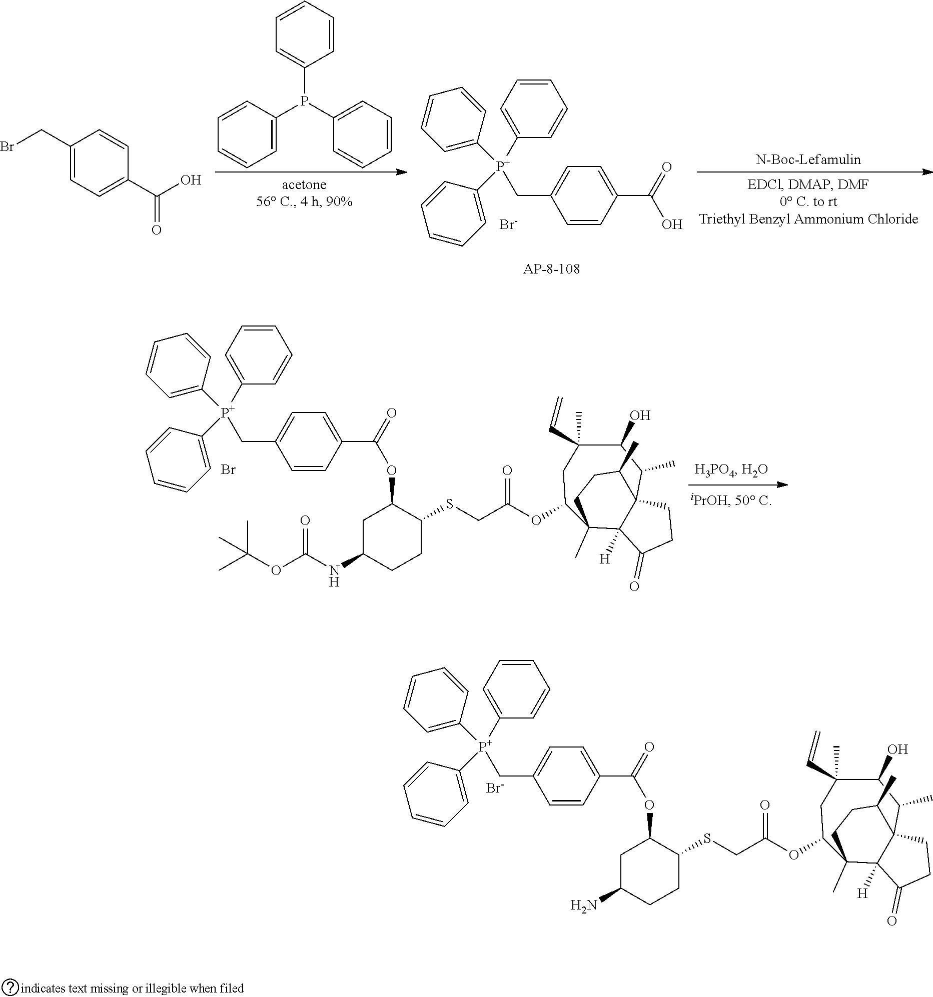

In order to facilitate development of the TPP containing antibiotics, a series of TPP linkers were prepared and tested for their toxicity in MRC5 cells. This library of linkers are shown below:

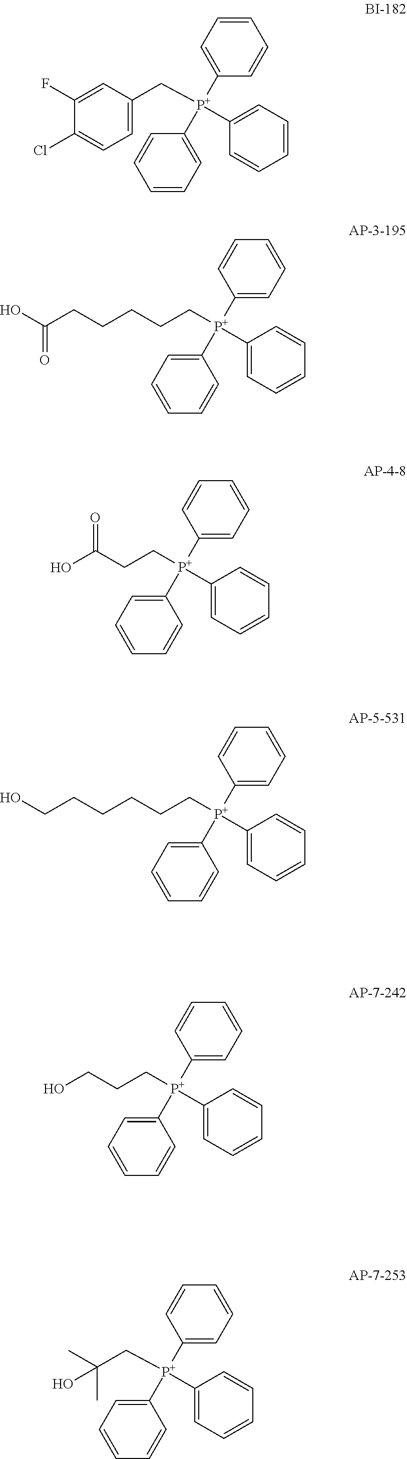 [SMILES: Fc1cc(C[P+](c2ccccc2)(c2ccccc2)c2ccccc2)ccc1Cl ; O=C(O)CCCCC[P+](c1ccccc1)(c1ccccc1)c1ccccc1 ; O=C(O)CC[P+](c1ccccc1)(c1ccccc1)c1ccccc1 ; OCCCCCC[P+](c1ccccc1)(c1ccccc1)c1ccccc1 ; OCCC[P+](c1ccccc1)(c1ccccc1)c1ccccc1 ; CC(C)(O)C[P+](c1ccccc1)(c1ccccc1)c1ccccc1]

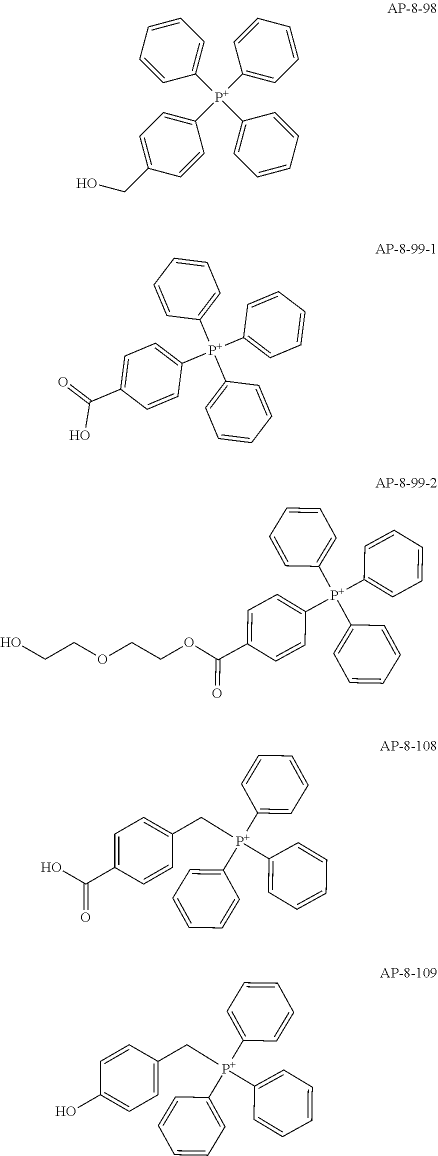 [SMILES: OCc1ccc([P+](c2ccccc2)(c2ccccc2)c2ccccc2)cc1 ; O=C(O)c1ccc([P+](c2ccccc2)(c2ccccc2)c2ccccc2)cc1 ; O=C(OCCOCCO)c1ccc([P+](c2ccccc2)(c2ccccc2)c2ccccc2)cc1 ; O=C(O)c1ccc(C[P+](c2ccccc2)(c2ccccc2)c2ccccc2)cc1 ; Oc1ccc(C[P+](c2ccccc2)(c2ccccc2)c2ccccc2)cc1]

The toxicity of these linkers in MRC5 cells after a 72 hour incubation. This data is shown in Table 1. Generally, these linkers showed very low toxicity with an IC₅₀ of greater than 90 μM with PS-341 (bortezomib) was used as a positive control.

TABLE 1
Cel Titer Glo 72-hour Incubation of TPP⁺ Linkers

| Compounds | IC₅₀, nM |
|---|---|
| BI-182 | >90000 |
| AP-3-195 | >90000 |
| AP-4-8 | >90000 |
| AP-5-531 | >90000 |
| AP-7-242 | >90000 |
| AP-7-253 | >90000 |
| AP-8-98 | 57628 |
| AP-8-99-1 | >90000 |
| AP-8-99-2 | >90000 |
| AP-8-108 | >90000 |
| AP-8-109 | 60574 |
| PS-341 | 7 |

The raw data for these linkers is shown in FIG. 3. Furthermore, the conjugate, AP-8-159B, shows weak cytoxcity in normal MRC5 fibroblast cell lines with PS-341 (bortezomib) was used as a positive control. This data is shown in Table 2 along with the raw data shown in FIG. 4.

TABLE 2
Cytotoxicity in MRC5 Fibroblast Cells of AP-8-159B

| Compounds | IC₅₀, nM |
|---|---|
| AP-8-159B | 4669 |
| Pleuromutilin CAS No: 125-65-5 | >90000 |
| AP-8-108 | >90000 |
| PS-341 | 0.3 |

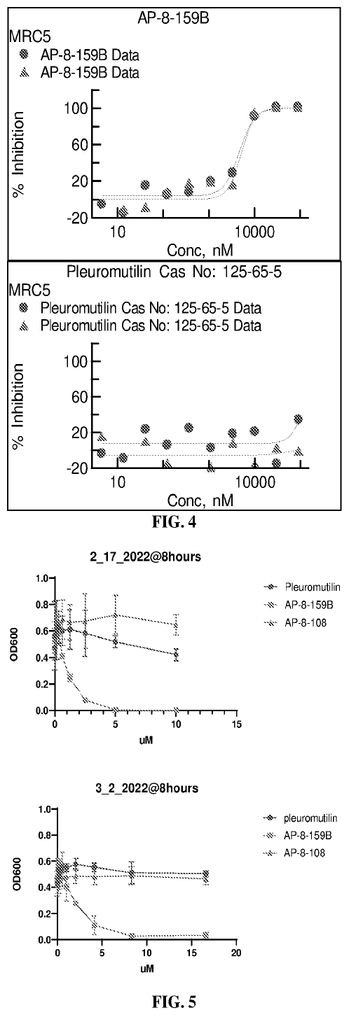

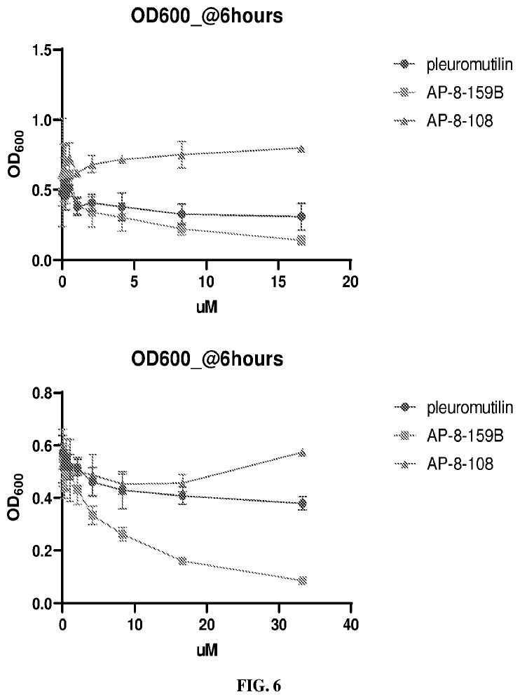

These compounds were tested in an E. coli and Klebsiella strain to determine the change in OD₆₀₀ as a function of concentration (FIG. 5, E. coli; FIG. 6, Klebsiella). The antibacterial activity of the compounds was further analyzed and activity was determine a variety of different bacterial strains. See Table 3 for more detail.

TABLE 3
Antibacterial in vitro activity of AP-8-159B compared to unconjugated Pleuromutilin and TPP⁺

| | | | MIC (μg/mL) | | | |
|---|---|---|---|---|---|---|
| | | | TPP+- | | | |
| | Isolate | Source/Genetic | Pleuromutilin | | | |
| Species | ID | information | AP-8-159B | Pleuromutilin | TPP+ | Doxycycline |
| Escherichia coli | B0001 | ATCC 25922 | 8 | 128 | >32 | 0.5 |
| Escherichia coli | D0120 | Clinical isolate | 16 | >128 | >32 | 2 |
| Escherichia coli | B1098 | Clinical isolate | 16 | >128 | >32 | 16 |
| Escherichia coli | B0158 | AcrAB-TolC | 32 | >128 | >32 | 2 |
| | | efflux pump WT | | | | |
| Escherichia coli | B0159 | Efflux pump | 0.5 | 0.5 | >32 | 0.25 |
| | | deficient (ΔtolC) | | | | |
| Escherichia coli | B0429 | Efflux pump | 1 | 32 | >32 | 0.25 |
| | | deficient (ΔacrAB) | | | | |
| Salmonella typhimurium | D0109 | ATCC13311 | 16 | 128 | ND | 0.5 |
| Citrobacter freundii | B0271 | Clinical isolate | 64 | >128 | ND | 8 |
| Citrobacter freundii | B0286 | AKH Vienna | 64 | >128 | ND | 2 |
| Klebsiella pneumoniae | D0033 | ATCC 29519 | 128 | >128 | ND | 1 |
| Klebsiella pneumoniae | B0305 | ATCC 43816 | 128 | >128 | ND | 1 |
| Klebsiella pneumoniae | B0032 | ATCC 11228 | >128 | >128 | ND | 0.25 |
| Enterobacter cloacae | D1090 | Clinical isolate | >128 | >128 | ND | 2 |
| Enterobacter cloacae | D1089 | Clinical isolate | >128 | >128 | ND | >32 |
| Proteus mirabilis | D1301 | Clinical isolate | 32 | >128 | ND | 32 |
| Proteus mirabilis | D1300 | Clinical isolate | 32 | >128 | ND | 32 |
| Acinetobacter baumannii | B0473 | ATCC 15473 | 64 | >128 | ND | 0.5 |
| Acinetobacter baumannii | B1062 | Clinical isolate | 128 | >128 | ND | ≤0.03 |
| Pseudomonas aeruginosa | B0016 | ATCC 29511 | 16 | >128 | ND | 32 |
| Pseudomonas aeruginosa | B0015 | ATCC 27853 | 8 | >128 | ND | 16 |
| Pseudomonas aeruginosa | B0367 | Clinical isolate | 4 | >128 | ND | >32 |
| Haemophilus influenzae | B0035 | ATCC 49247 | 1 | 4 | ND | 0.12 |
| Haemophilus influenzae | B0126 | Clinical isolate | 0.25 | 1 | ND | 0.12 |
| Staphylococcus aureus | B0009 | ATCC 49951 | ≤0.12 | 1 | ND | 0.12 |
| Staphylococcus aureus | B0029 | Clinical isolate | 0.5 | 1 | ND | 8 |
| Staphylococcus aureus | B1325 | Clinical isolates | 0.5 | ND | ND | 4 |
| Enterococcus faecium | D0459 | Clinical isolate | 0.5 | ND | ND | 32 |
| Streptococcus pneumoniae | B0415 | Clinical isolate | ≤0.12 | 1 | ND | 0.12 |
| Streptococcus pneumoniae | B0078 | Clinical isolate | 0.5 | 4 | ND | 16 |
| Streptococcus pyogenes | B0780 | Clinical isolate | 0.12 | ND | ND | 16 |
| Streptococcus agalactiae | B0790 | Clinical isolate | 0.06 | ND | ND | 0.12 |

All of the compositions and/or methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and/or methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference:
Barnoud et al., Cancer Res., 80(23):5270-81, 2020.
Costa et al., Antibiotics (Basel)., 10(8):974, 2021.
Eyal et al., Sci Rep. 6:39004, 2016.
Gentry et al., Antimicrob Agents Chemother., 51(6):2048-52, 2007.
Grinius et al., Biochim Biophys Acta, 216(1):1-12, 1970.
Högenauer & Ruf, Antimicrob Agents Chemother., 19(2):260-5, 1981.
Högenauer, Eur J Biochem., 52(1):93-8, 1975.
Jeong et al., Genome Announc., 3(2):e00134-15, 2015.
Kavanagh et al., Proc Natl Acad Sci USA., 37(9):570-4, 1951.
Long et al., Antimicroh Agents Chemother., 50(7):2500-5, 2006.
May & Grabowicz, Proc Natl Acad Sci USA., 115(36):8852-54, 2018.
McCarthy, Clin Pharmacokinet., Ahead of print, 2021
Murphy, Biochim Biophys Acta., 1777(7-8):1028-31, 2008.
Nikaido, Clin Infect Dis., Suppl 1:S32-41, 1998.
Nikaido, Microbiol Mol Biol Rev., 67(4):593-656, 2003.
Paukner & Riedl, Cold Spring Harb PerspectMed., 7(1):a027110, 2017.
Prestinaci et al., Pathog Glob Health., 109(7):309-18, 2015.
Toté et al., J Appl Microbiol. 107(2):606-15, 2009.
Travnickova et al., AMB Express., 9(1):183, 2019.
Wannigama et al., Sci Rep., 9(1):6300, 2019.
Yarlagadda et al., ACS Infect Dis. 2(2):132-9, 2016.
Zgurskaya & Nikaido, Mol Microbiol., 37(2):219-25, 2000.

What is claimed:

1. A compound comprising a triaryl phosphonium cation and an antibiotic.

2. The compound of claim 1, wherein the compound further comprises a linker.

3. The compound of either claim 1 or claim 2, wherein the antibiotic is pleuromutilin or a pleuromutilin analog.

4. The compound according to any one of claims 1-3, wherein the compound is further defined as: 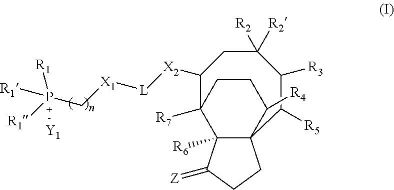
wherein:
R₁, R₁′, and R₁″ are each independently aryl_{(C≤12)} or substituted aryl_{(C≤12)};
n is 0, 1, 2, or 3;
Y₁ is a monovalent anion;
X₁ and X₂ are each conjugating groups;
L is a linker group;
R₂ and R₂′ are hydrogen, alkyl_{(C≤12)}, alkenyl_{(C≤12)}, aryl_{(C≤12)}, aralkyl_{(C≤12)}, or a substitute version thereof;
R₃ is hydrogen, hydroxy, alkoxy_{(C≤12)}, substituted alkoxy_{(C≤12)}, acyloxy_{(C≤12)}, or substituted acyloxy_{(C≤12)};
R₄, R₅, R₆, and R₇ are each independently are selected from hydrogen, alkyl_{(C≤12)}, or substituted alkyl_{(C≤12)}; and
Z is O, S, or NRₐ, wherein Rₐ is hydrogen, alkyl_{(C≤6)}, or substituted alkyl_{(C≤6)};
or a pharmaceutically acceptable salt thereof.

5. The compound of claim 4 further defined as: 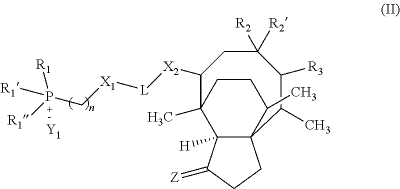
wherein:
R₁, R₁′, and R₁″ are each independently aryl_{(C≤12)} or substituted aryl_{(C≤12)};
n is 0, 1, 2, or 3;
Y₁ is a monovalent anion;
X₁ and X₂ are each conjugating groups;
L is a linker group;
R₂ and R₂′ are hydrogen, alkyl_{(C≤12)}, alkenyl_{(C≤12)}, aryl_{(C≤12)}, aralkyl_{(C≤12)}, or a substitute version thereof;
R₃ is hydrogen, hydroxy, alkoxy_{(C≤12)}, substituted alkoxy_{(C≤12)}, acyloxy_{(C≤12)}, or substituted acyloxy_{(C≤12)}; and
Z is O, S, or NRₐ, wherein Rₐ is hydrogen, alkyl_{(C≤6)}, or substituted alkyl_{(C≤6)};
or a pharmaceutically acceptable salt thereof.

6. The compound of either claim 4 or claim 5 further defined as: 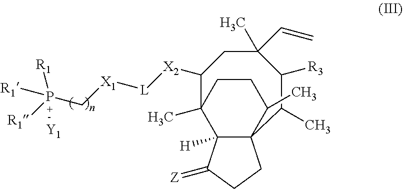
wherein:
R₁, R₁′, and R₁″ are each independently aryl_{(C≤12)} or substituted aryl_{(C≤12)};
n is 0, 1, 2, or 3;
Y₁ is a monovalent anion;
X₁ and X₂ are each conjugating groups;
L is a linker group;
R₃ is hydrogen, hydroxy, alkoxy_{(C≤12)}, substituted alkoxy_{(C≤12)}, acyloxy_{(C≤12)}, or substituted acyloxy_{(C≤12)}; and
Z is O, S, or NRₐ, wherein Rₐ is hydrogen, alkyl_{(C≤6)}, or substituted alkyl_{(C≤6)};
or a pharmaceutically acceptable salt thereof.

7. The compound according to any one of claims 4-6 further defined as: 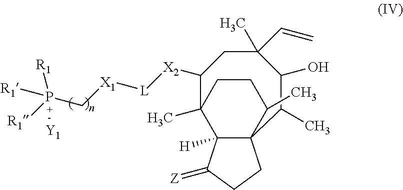
wherein:
R₁, R₁′, and R₁″ are each independently aryl_{(C≤12)} or substituted aryl_{(C≤12)};
n is 0, 1, 2, or 3;
Y₁ is a monovalent anion;
X₁ and X₂ are each conjugating groups;
L is a linker group; and
Z is O, S, or NRₐ, wherein Rₐ is hydrogen, alkyl_{(C≤6)}, or substituted alkyl_{(C≤6)};
or a pharmaceutically acceptable salt thereof.

8. The compound of claim 4 further defined as: 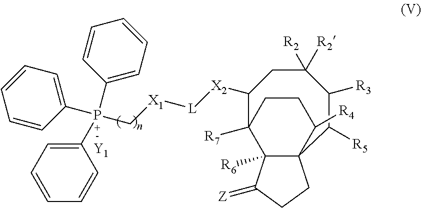
wherein:
n is 0, 1, 2, or 3;
Y₁ is a monovalent anion;
X₁ and X₂ are each conjugating groups;
L is a linker group;
R₂ and R₂′ are hydrogen, alkyl_{(C≤12)}, alkenyl_{(C≤12)}, aryl_{(C≤12)}, aralkyl_{(C≤12)}, or a substitute version thereof;
R₃ is hydrogen, hydroxy, alkoxy_{(C≤12)}, substituted alkoxy_{(C≤12)}, acyloxy_{(C≤12)}, or substituted acyloxy_{(C≤12)};
R₄, R₅, R₆, and R₇ are each independently are selected from hydrogen, alkyl_{(C≤12)}, or substituted alkyl_{(C≤12)}; and
Z is O, S, or NRₐ, wherein Rₐ is hydrogen, alkyl_{(C≤6)}, or substituted alkyl_{(C≤6)};
or a pharmaceutically acceptable salt thereof.

9. The compound according to any one of claim 4-8 further defined: 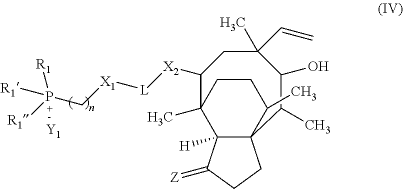
wherein:
R₁, R₁′, and R₁″ are each independently aryl_{(C≤12)} or substituted aryl_{(C≤12)};
n is 0, 1, 2, or 3;
Y₁ is a monovalent anion;
X₁ and X₂ are each conjugating groups;
L is a linker group; and
Z is O, S, or NRₐ, wherein Rₐ is hydrogen, alkyl_{(C≤6)}, or substituted alkyl_{(C≤6)};
or a pharmaceutically acceptable salt thereof.

10. The compound according to any one of claims 4-9 further defined as: 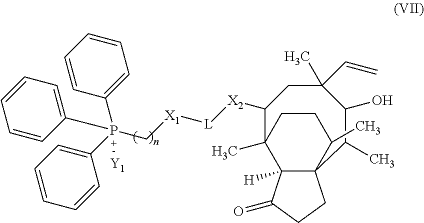
wherein:
n is 0, 1, 2, or 3;
Y₁ is a monovalent anion;
X₁ and X₂ are each conjugating groups; and
L is a linker group;
or a pharmaceutically acceptable salt thereof.

11. The compound according to any one of claims 4-10 further defined as: 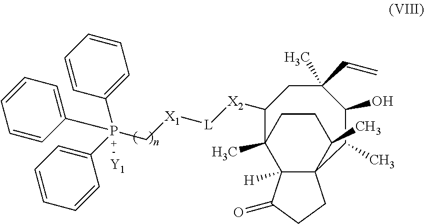
wherein:
n is 0, 1, 2, or 3;
Y₁ is a monovalent anion;
X₁ and X₂ are each conjugating groups; and
L is a linker group;
or a pharmaceutically acceptable salt thereof.

12. The compound of either claim 4 or claim 8, wherein R₄ is alkyl_{(C≤12)} or substituted alkyl_{(C≤12)}.

13. The compound of claim 12, wherein R₄ is alkyl_{(C≤12)}.

14. The compound of either claim 12 or claim 13, wherein R₄ is methyl.

15. The compound according to any one of claims 4, 8, and 12-14, wherein R₅ is alkyl_{(C≤12)} or substituted alkyl_{(C≤12)}.

16. The compound of claim 15, wherein R₅ is alkyl_{(C≤12)}.

17. The compound of either claim 15 or claim 16, wherein R₅ is methyl.

18. The compound according to any one of claims 4, 8, and 12-17, wherein R₆ is hydrogen.

19. The compound according to any one of claims 4, 8, and 12-18, wherein R₇ is alkyl_{(C≤12)} or substituted alkyl_{(C≤12)}.

20. The compound of claim 19, wherein R₇ is alkyl_{(C≤12)}.

21. The compound of either claim 19 or claim 20, wherein R₇ is methyl.

22. The compound according to any one of claims 4, 5, 8, and 12-21, wherein R₂ is alkyl_{(C≤12)} or substituted alkyl_{(C≤12)}.

23. The compound of claim 22, wherein R₂ is alkyl_{(C≤12)}.

24. The compound of either claim 22 or claim 23, wherein R₂ is methyl.

25. The compound according to any one of claims 4, 5, 8, and 12-24, wherein R₂′ is alkenyl_{(C≤12)} or substituted alkenyl_{(C≤12)}.

26. The compound of claim 25, wherein R₂′ is alkenyl_{(C≤12)}.

27. The compound of either claim 25 or claim 26, wherein R₂′ is ethenyl.

28. The compound according to any one of claims 4-6, 8, and 12-27, wherein R₃ is hydroxy.

29. The compound according to any one of claims 4-9 and 12-28, wherein Z is O.

30. The compound according to any one of claims 4-7, 9, and 12-29, wherein R₁ is aryl_{(C≤12)} or substituted aryl_{(C≤12)}.

31. The compound of claim 30, wherein R₁ is aryl_{(C≤12)}.

32. The compound of claim 31, wherein R₁ is phenyl.

33. The compound according to any one of claims 4-7, 9, and 12-32, wherein R₁′ is aryl_{(C≤12)} or substituted aryl_{(C≤12)}.

34. The compound of claim 33, wherein R₁′ is aryl_{(C≤12)}.

35. The compound of claim 34, wherein R₁′ is phenyl.

36. The compound according to any one of claims 4-7, 9, and 12-35, wherein R₁″ is aryl_{(C≤12)} or substituted aryl_{(C≤12)}.

37. The compound of claim 36, wherein R₁″ is aryl_{(C≤12)}.

38. The compound of claim 37, wherein R₁″ is phenyl.

39. The compound according to any one of claims 4-38, wherein n is 0, 1, or 2.

40. The compound of claim 39, wherein n is 0.

41. The compound of claim 39, wherein n is 1.

42. The compound of claim 39, wherein n is 2.

43. The compound according to any one of claims 4-42, wherein Y₁ is a halide.

44. The compound of claim 43, wherein Y₁ is bromide or chloride.

45. The compound according to any one of claims 4-44, wherein X₁ is a conjugating group selected from: alkanediyl_{(C≤12)}, substituted alkanediyl_{(C≤12)}, alkenediyl_{(C≤12)}, substituted alkenediyl_{(C≤12)}, arenediyl_{(C≤12)}, substituted arenediyl_{(C≤12)}, —C(O)—, —C(O)O—, —OC(O)—, —C(O)NR_{b}—, —NR_{b}C(O)—, —OC(O)O—, —NR_{b}C(O)NR_{b}—, —S(O)ₓ—, —OS(O)ₓO—, —OS(O)ₓ—, —S(O)ₓO—, and a combination thereof;
wherein: x is 0, 1, or 2; and R_{b} is hydrogen, alkyl_{(C≤6)}, or substituted alkyl_{(C≤6)}.

46. The compound of claim 45, wherein X₁ is a conjugating group selected from: alkanediyl_{(C≤12)}, substituted alkanediyl_{(C≤12)}, arenediyl_{(C≤12)}, substituted arenediyl_{(C≤12)}, —C(O)—, —C(O)O—, —OC(O)—, —C(O)NR_{b}—, —NR_{b}C(O)—, and a combination thereof.

47. The compound of claim 46, wherein X₁ is a conjugating group is a group comprising an arenediyl_{(C≤12)} or substituted arenediyl_{(C≤12)} and —C(O)O— or —OC(O)—.

48. The compound of claim 47, wherein X₁ is an arenediyl_{(C≤12)} and —C(O)O—.

49. The compound of claim 48, wherein X₁ is benzenediyl and —C(O)O—.

50. The compound of claim 49, wherein X₁ is —C₆H₄—C(O)O—.

51. The compound according to any one of claims 4-50, wherein X₂ is a conjugating group selected from: alkanediyl_{(C≤12)}, substituted alkanediyl_{(C≤12)}, alkenediyl_{(C≤12)}, substituted alkenediyl_{(C≤12)}, arenediyl_{(C≤12)}, substituted arenediyl_{(C≤12)}, —C(O)—, —C(O)O—, —OC(O)—, —C(O)NR_{b}—, —NR_{b}C(O)—, —OC(O)O—, —NR_{b}C(O)NR_{b}—, —S(O)ₓ—, —OS(O)ₓO—, —OS(O)ₓ—, —S(O)ₓO—, and a combination thereof;
wherein: x is 0, 1, or 2; and R_{b} is hydrogen, alkyl_{(C≤6)}, or substituted alkyl_{(C≤6)}.

52. The compound of claim 51, wherein X₂ is a conjugating group selected from: —C(O)—, —C(O)O—, —OC(O)—, —C(O)NR_{b}—, —NR_{b}C(O)—, —OC(O)O—, and —NR_{b}C(O)NR_{b}—.

53. The compound of claim 52, wherein X₂ is a conjugating group selected from: —C(O)—, —C(O)O—, —OC(O)—, and —OC(O)O—.

54. The compound of claim 53, wherein X₂ is —C(O)O— or —OC(O)—.

55. The compound according to any one of claims 4-54, wherein L is a linking group selected from: a covalent bond, alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, alkynediyl_{(C≤12)}, substituted alkynediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, a polypeptide comprising from about 1-100 amino acids, a polyethylene oxide, a polypropylene oxide, a methacrylate polymer, a polyester polymer, a polyamide, a polyamine, —NR_{c}—, —O—, —S—, —C(O)—, —C(O)O—, —OC(O)—, —C(O)NR_{c}—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof;
wherein: y is 0, 1, or 2; and R_{c} is hydrogen, alkyl_{(C≤6)}, or substituted alkyl_{(C≤6)}.

56. The compound of claim 55, wherein L is a linking group selected from: a covalent bond, alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, alkynediyl_{(C≤12)}, substituted alkynediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, or a combination thereof.

57. The compound of claim 56, wherein L is a linking group selected from: a covalent bond, alkanediyl_{(C≤12)}, substituted alkanediyl_{(C≤12)}, cycloalkanediyl_{(C≤12)}, substituted cycloalkanediyl_{(C≤12)}, arenediyl_{(C≤12)}, substituted arenediyl_{(C≤12)}, or a combination thereof.

58. The compound of claim 57, wherein L is a linker group selected from: a covalent bond, alkanediyl_{(C≤12)}, or substituted alkanediyl_{(C≤12)}.

59. The compound of claim 58, wherein L is alkanediyl_{(C≤12)} or substituted alkanediyl_{(C≤12)}.

60. The compound of claim 59, wherein L is alkanediyl_{(C≤12)}.

61. The compound of claim 60, wherein L is —CH₂—.

62. The compound of claim 56, wherein L is a linker group selected from: a covalent bond, alkanediyl_{(C≤12)}, substituted alkanediyl_{(C≤12)}, cycloalkanediyl_{(C≤12)}, substituted cycloalkanediyl_{(C≤12)}, heterocycloalkanediyl_{(C≤12)}, substituted heterocycloalkanediyl_{(C≤12)}, —NR_{c}—, —O—, —S—, —C(O)—, —C(O)O—, —OC(O)—, —C(O)NR_{c}—, —NR_{c}C(O)—, —OC(O)O—, —NR_{c}C(O)NRₑ—, —S(O)_{y}—, —OS(O)_{y}O—, —OS(O)_{y}—, —S(O)_{y}O—, or a combination thereof.

63. The compound of claim 62, wherein L is a linker group selected from: alkanediyl_{(C≤12)}, substituted alkanediyl_{(C≤12)}, cycloalkanediyl_{(C≤12)}, substituted cycloalkanediyl_{(C≤12)}, heterocycloalkanediyl_{(C≤12)}, substituted heterocycloalkanediyl_{(C≤12)}, —NR_{c}—, —O—, —S—, or a combination thereof.

64. The compound of claim 63, wherein L comprises an alkanediyl_{(C≤12)}, substituted alkanediyl_{(C≤12)}, a cycloalkanediyl_{(C≤12)}, substituted cycloalkanediyl_{(C≤12)}, and —S—.

65. The compound of claim 64, wherein L is —X₃—X₄—X₅—;
wherein:
X₃ is cycloalkandiyl_{(C≤12)} or substituted cycloalkanediyl_{(C≤12)};
X₄ is —S—;
X₅ is alkanediyl_{(C≤12)} or substituted alkanediyl_{(C≤12)}.

66. The compound of claim 65, wherein X₃ is substituted cycloalkanediyl_{(C≤12)}.

67. The compound of claim 66, wherein X₃ is 4-aminocyclohexanediyl.

68. The compound according to any one of claims 65-67, wherein X₅ is alkanediyl_{(C≤12)}.

69. The compound of claim 68, wherein X₅ is methylene.

70. The compound according to any one of claims 1-69, wherein the compound is further defined as: 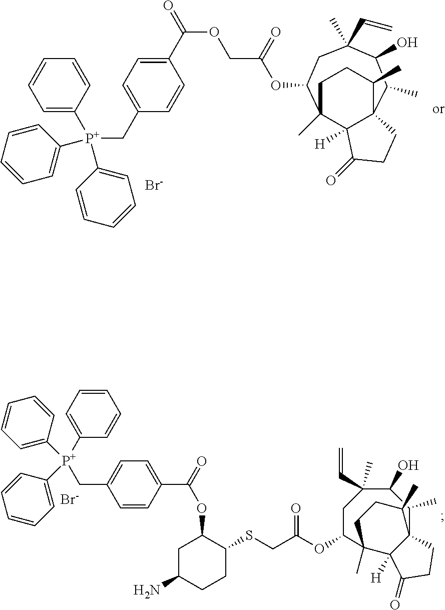
or a pharmaceutically acceptable salt thereof.

71. A compound of the formula: 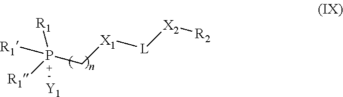
wherein:
R₁, R₁′, and R₁″ are each independently aryl_{(C≤12)} or substituted aryl_{(C≤12)};
n is 0, 1, 2, or 3;
Y₁ is a monovalent anion;
X₁ and X₂ are each independently conjugating groups;
L is a linker group; and
R₂ is a fluorophore;
or a pharmaceutically acceptable salt thereof.

72. The compound of claim 71, wherein R₁, R₁′, and R₁″ are each the same aryl_{(C≤12)} or substituted aryl_{(C≤12)}.

73. The compound of claim 72, wherein R₁, R₁′, and R₁″ are each phenyl.

74. The compound according to any one of claims 71-73, wherein n is 0 or 1.

75. The compound according to any one of claims 71-74, wherein L is a covalent bond.

76. The compound according to any one of claims 71-75, wherein X₁ is alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, alkynediyl_{(C≤12)}, substituted alkynediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, a polypeptide comprising from about 1-100 amino acids, a polyethylene oxide, a polypropylene oxide, a methacrylate polymer, a polyester polymer, a polyamide, a polyamine, —NR_{c}—, —O—, —S—, —C(O)—, —C(O)O—, —OC(O)—, —C(O)NR_{c}—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof;
wherein: y is 0, 1, or 2; and R_{c} is hydrogen, alkyl_{(C≤6)}, or substituted alkyl_{(C≤6)}.

77. The compound of claim 76, wherein X₁ is, —NR_{c}—, —O—, —S—, —C(O)—, —C(O)O—, —OC(O)—, —C(O)NR_{c}—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof;
wherein: y is 0, 1, or 2; and R_{c} is hydrogen, alkyl_{(C≤6)}, or substituted alkyl_{(C≤6)}.

78. The compound of claim 77, wherein X₁ is —C(O)—.

79. The compound according to any one of claims 71-78, wherein X₂ is alkanediyl_{(C≤12)}, substituted alkanediyl_{(C≤12)}, cycloalkanediyl_{(C≤12)}, substituted cycloalkanediyl_{(C≤12)}, alkenediyl_{(C≤12)}, substituted alkenediyl_{(C≤12)}, alkynediyl_{(C≤12)}, substituted alkynediyl_{(C≤12)}, arenediyl_{(C≤12)}, substituted arenediyl_{(C≤12)}, heteroarenediyl_{(C≤12)}, substituted heteroarenediyl_{(C≤12)}, heterocycloalkanediyl_{(C≤12)}, substituted heterocycloalkanediyl_{(C≤12)}, a polypeptide comprising from about 1-100 amino acids, a polyethylene oxide, a polypropylene oxide, a methacrylate polymer, a polyester polymer, a polyamide, a polyamine, —NR_{c}—, —O—, —S—, —C(O)—, —C(O)O—, —OC(O)—, —C(O)NR_{c}—, —NR_{c}C(O)—, —OC(O)O—, —NR_{c}C(O)NRₑ—, —S(O)_{y}—, —OS(O)_{y}O—, —OS(O)_{y}—, —S(O)_{y}O—, or a combination thereof;
wherein: y is 0, 1, or 2; and R_{c} is hydrogen, alkyl_{(C≤6)}, or substituted alkyl_{(C≤6)}.

80. The compound of claim 79, wherein X₂ is, —NR_{c}—, —O—, —S—, —C(O)—, —C(O)O—, —OC(O)—, —C(O)NRₑ—, —NR_{c}C(O)—, —OC(O)O—, —NR_{c}C(O)NR_{c}—, —S(O)_{y}—, —OS(O)_{y}O—, —OS(O)_{y}—, —S(O)_{y}O—, or a combination thereof;
wherein: y is 0, 1, or 2; and R_{c} is hydrogen, alkyl_{(C≤6)}, or substituted alkyl_{(C≤6)}.

81. The compound of claim 80, wherein X₂ is —O—.

82. The compound according to any one of claims 71-81 further defined as: 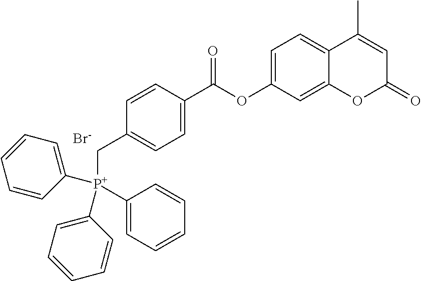 [SMILES: Cc1cc(=O)oc2cc(OC(=O)c3ccc(C[P+](c4ccccc4)(c4ccccc4)c4ccccc4)cc3)ccc12 ; [Br-]]
or a pharmaceutically acceptable salt thereof.

83. A pharmaceutical composition comprising:
(A) a compound according to any one of claims 1-82; and
(B) an excipient.

84. The pharmaceutical composition of claim 83, wherein the pharmaceutical composition is formulated for administration: orally, intraadiposally, intraarterially, intraarticularly, intracranially, intradermally, intralesionally, intramuscularly, intranasally, intraocularly, intrapericardially, intraperitoneally, intrapleurally, intraprostatically, intrarectally, intrathecally, intratracheally, intratumorally, intraumbilically, intravaginally, intravenously, intravesicularlly, intravitreally, liposomally, locally, mucosally, parenterally, rectally, subconjunctival, subcutaneously, sublingually, topically, transbuccally, transdermally, vaginally, in cremes, in lipid compositions, via a catheter, via a lavage, via continuous infusion, via infusion, via inhalation, via injection, via local delivery, or via localized perfusion.

85. The pharmaceutical composition of either claim 83 or claim 84, wherein the pharmaceutical composition is formulated as a unit dose.

86. A method of treating a disease or disorder in a patient in need thereof comprising administering a therapeutically effective amount of a compound or composition according to any one of claims 1-85.

87. The method of claim 86, wherein the disease or disorder is an infection.

88. The method of claim 87, wherein the infection is a bacterial infection.

89. The method of claim 88, wherein the bacterial infection is an infection of a gram positive bacteria.

90. The method of claim 89, wherein the gram positive bacteria is a Staphylococcus bacteria.

91. The method of claim 90, wherein the Staphylococcus bacteria is a Staphylococcus aureus bacteria.

92. The method of claim 88, wherein the bacterial infection is an infection of a gram negative bacteria.

93. The method of claim 92, wherein the gram negative bacteria is an Escherichia bacteria.

94. The method of claim 93, wherein the Escherichia bacteria is Escherichia coli bacteria.

95. The method of claim 92, wherein the gram negative bacteria is an Klebsiella bacteria.

96. The method of claim 95, wherein the Escherichia bacteria is Klebsiella pneunoniae bacteria.

97. The method according to any one of claims 86-96, wherein the method further comprises administering a second therapeutic agent.

98. The method of claim 97, wherein the second therapeutic agent is an antibiotic.

99. The method according to any one of claims 86-98, wherein the method comprises administering the compound or composition once.

100. The method according to any one of claims 86-98, wherein the method comprises administering the compound or composition two or more times.

101. A method of inhibiting the growth of a bacterium comprising contacting the bacterium with a compound or composition according to any one of claims 1-85.

102. The method of claim 101, wherein the method is performed in vitro.

103. The method of claim 101, wherein the method is performed in vivo.

104. The method according to any one of claims 101-103, wherein the bacterium is a gram positive bacterium.

105. The method of claim 104, wherein the gram positive bacterium is a Staphylococcus bacterium.

106. The method of claim 105, wherein the Staphylococcus bacterium is a Staphylococcus aureus bacterium.

107. The method according to any one of claims 101-103, wherein the bacterium is a gram negative bacterium.

108. The method of claim 107, wherein the gram negative bacterium is an Escherichia bacterium.

109. The method of claim 108, wherein the Escherichia bacterium is Escherichia coli bacterium.

110. The method of claim 107, wherein the gram negative bacteria is an Klebsiella bacteria.

111. The method of claim 110, wherein the Escherichia bacteria is Klebsiella pneumoniae bacteria.

112. A method of killing a bacterium comprising contacting the bacterium with a compound or composition according to any one of claims 1-85.

113. The method of claim 112, wherein the method is performed in vitro.

114. The method of claim 112, wherein the method is performed in vivo.

115. The method according to any one of claims 112-114, wherein the bacterium is a gram positive bacterium.

116. The method of claim 115, wherein the gram positive bacterium is a Staphylococcus bacterium.

117. The method of claim 116, wherein the Staphylococcus bacterium is a Staphylococcus aureus bacterium.

118. The method according to any one of claims 112-114, wherein the bacterium is a gram negative bacterium.

119. The method of claim 118, wherein the gram negative bacterium is an Escherichia bacterium.

120. The method of claim 119, wherein the Escherichia bacterium is Escherichia coli bacterium.

121. The method of claim 118, wherein the gram negative bacteria is an Klebsiella bacteria.

122. The method of claim 121, wherein the Escherichia bacteria is Klebsiella pneunoniae bacteria.
